(19) United States
(12) Patent Application Publication
(10) Pub. No.: US 2017/0115310 A1
(43) Pub. Date: Apr. 27, 2017

(54) PREDICTING RAPID DECLINE IN RENAL FUNCTION IN DIABETES

(71) Applicant: UNIVERSITY OF DUNDEE, Dundee (GB)

(72) Inventors: Helen COLHOUN, Dundee (GB); Helen LOOKER, Dundee (GB); Paul MCKEIGUE, Edinburgh (GB); Marco COLUMBO, Edinburgh (GB); David DUNGER, Cambridge (GB); Neil DALTON, London (GB); Mary Julia BROSNAN, Cambridge, MA (US); Everson NOGOCEKE, Basel (CH); Felix AGAKOV, Edinburgh (GB); Sibylle HESS, Frankfurt am Main (DE)

(21) Appl. No.: 15/126,677

(22) PCT Filed: Mar. 18, 2015
(86) PCT No.: PCT/GB2015/050797

§ 371 Date: Sep. 16, 2016

(30) Foreign Application Priority Data
Mar. 18, 2014 (GB) 1404789.8
Nov. 25, 2014 (GB) 1420908.4

(51) Int. Cl. G01N 33/68; G01N 33/50; G06F 19/00; G01N 33/70

(52) U.S. Cl. CPC G01N 33/6893; G01N 33/70; G01N 33/50; G06F 19/3431; G01N 2800/347; G01N 2800/50; G01N 2800/52

(57) ABSTRACT

Methods of predicting rapid decline in renal function in diabetes employing panels of biomarkers. The methods are useful for predicting risk of decline of renal function in individual subject and also for selecting subjects for clinical trials.

### FIELD OF THE INVENTION

The invention concerns the prediction of rapid decline in renal function in human patients suffering from diabetes and applications of that prediction, for example to the selection of subjects for clinical trials for renal function decline arising from diabetes.

### BACKGROUND TO THE INVENTION

Renal disease is an important complication of diabetes resulting in significant morbidity and mortality. In the UK the prevalence of end stage renal disease (ESRD) due to diabetes has more than doubled in the past decade (Hill C J, Fogarty D G. Changing trends in end-stage renal disease due to diabetes in the United kingdom. J Ren Care. 2012; 38 Suppl 1: 12-22) and while there is evidence that the rate of incident ESRD due to diabetes is now slowing or even declining in some studies, it remains a major health issue for people with diabetes as well as a cost to the health service (Couchoud C, Villar E. End-stage renal disease epidemic in diabetics: is there light at the end of the tunnel? Nephrol Dial Transplant. 2013; 28(5): 1073-6).

Albuminuria is a key measure of diabetic nephropathy progression but renal function loss is not always accompanied by proteinuria, and in the UK Prospective Diabetes Study (UKPDS) 51% of people who developed chronic kidney disease stage 3 (Key Facts and Figures. 2010; available from: www.kidneycare.nhs.uk/document.php?o=476) (CKD3) or worse had no history of albuminuria (Retnakaran R, Cull C A, Thorne K I, Adler A I, Holman R R. Risk factors for renal dysfunction in type 2 diabetes: U.K. Prospective Diabetes Study 74. Diabetes. 2006; 55(6): 1832-9). Thus, we have focused on decline in renal function itself as assessed by estimated glomerular filtration rate (eGFR) rather than progression of albuminuria.

Whilst identifying individuals at high risk for progression of renal dysfunction is important clinically it is also vital for drug companies running trials for treatments aimed at slowing progression of renal disease. These trials are powered by the number of individuals with rapid progression of renal dysfunction and as this is not easily predicted at present trials need to recruit large number of people and follow them for many years, both of which add to the cost of these trials. A key challenge for clinical trials of agents to prevent chronic kidney disease (CKD) progression (either to an endpoint of some given eGFR or to ESRD) in diabetes is that many people even with reduced eGFR remain stable over fairly long periods of time whereas some others will progress to ESRD or lose eGFR much more rapidly. An important goal is therefore to be able to identify the subset of people with rapid progression. The invention addresses this by considering both the level of biomarkers and clinical covariates and we have identified a panel of biomarkers that can be used to improve prediction of risk for renal dysfunction in people with diabetes.

Measures of eGFR and albumin excretion are routine for assessing diabetic nephropathy status in clinical practice, however new biomarkers to indicate people at greatest risk for rapid progression could improve management. In addition, identifying individuals at high risk for progression of renal dysfunction is also vital for drug companies running trials for treatments aimed at slowing progression of renal disease. These trials are powered by the number of individuals with rapid progression of renal dysfunction and as this is not easily predicted at present trials need to recruit large number of people and follow them for many years, both of which add to the cost of these trials. Thus ways to better predict individuals at high risk for progression over a relatively short time period (3-4 years) would greatly reduce the cost required for such trials and some aspects of the invention aim to identify the subset of people with rapid progression in order that they can be selected as participants in clinical trials.

### SUMMARY OF THE INVENTION

According to a first aspect of the invention there is provided a method of determining renal function decline risk in a (human) subject, comprising (a) analysing one or more samples obtained from the subject for the level of each of a plurality of biomarkers, (b) comparing the measured levels of each of the plurality of biomarkers with a respective plurality of control levels, and (c) determining renal function decline risk in the subject from the comparison, wherein the said plurality of biomarkers comprise at least 7 of the group consisting of: (i) adrenomedullin; (ii) alpha-1 antitrypsin; (iii) beta-2-microglobulin; (iv) c16-acylcarnitine; (v) creatine; (vi) creatinine; (vii) fatty acid-binding protein, heart; (viii) fibroblast growth factor 21; (ix) hydroxyproline; (x) kidney injury molecule-1; (xi) N-terminal prohormone of brain natriuretic peptide; (xii) the ratio of symmetric dimethylarginine to asymmetric dimethylarginine; (xiii) symmetric dimethylarginine; and (xiv) uracil.

It may be that step (c) also comprises taking into account each of a plurality of clinical covariates pertaining to the subject, wherein the clinical covariates comprise at least 5 of the group consisting of: (a) the age of the subject; (b) the gender of the subject; (c) an estimate of glomerular filtration rate (eGFR) of the subject; (d) the level of albumin in urine from the patient; (e) the level of HbA1c in one or more samples obtained from the subject; (f) a measure of the use of ACE inhibitors by the subject; (g) a measure of the use of angiotensin receptor blocker use by the subject.

According to a second aspect of the invention there is provided a method of selecting a (human) subject as a candidate subject for a study concerning the efficacy of a method of treatment on renal function or the progression of decline in renal function, comprising (a) analysing one or more samples obtained from the subject for the level of each of a plurality of biomarkers, (b) comparing the measured levels of each of the plurality of biomarkers with a respective plurality of control levels, (c) determining whether to select the subject as a candidate subject for the study in dependence on the comparison, wherein the said plurality of biomarkers comprise at least 7 of the group consisting of: (i) adrenomedullin; (ii) alpha-1 antitrypsin; (iii) beta-2-microglobulin; (iv) c16-acylcarnitine; (v) creatine; (vi) creatinine; (vii) fatty acid-binding protein, heart; (viii) fibroblast growth factor 21; (ix) hydroxyproline; (x) kidney injury molecule-1; (xi) N-terminal prohormone of brain natriuretic peptide; (xii) the ratio of symmetric dimethylarginine to asymmetric dimethylarginine; (xiii) symmetric dimethylarginine; and (xiv) uracil.

It may be that step (c) also comprises taking into account each of a plurality of clinical covariates pertaining to the subject, wherein the clinical covariates comprise at least 5 of the group consisting of: (a) the age of the subject; (b) the gender of the subject; (c) an estimate of glomerular filtration rate (eGFR) of the subject; (d) the level of albumin in urine from the patient; (e) the level of HbA1c in one or more samples obtained from the subject; (f) a measure of the use of ACE inhibitors by the subject; (g) a measure of the use of angiotensin receptor blocker use by the subject.

Typically, subjects who are determined from the said comparison to be have a risk of decline of renal function exceeding a threshold are selected as candidate subjects. A lower threshold might be used to obtain larger groups of candidate subjects and a high threshold to obtain a smaller group of candidate subjects at greatest risk. The level of the threshold can be set depending on the desired risk level of candidate subjects, for example to obtain those in the top 25%, top 10% or top 5% of a group of subjects. A candidate subject may be selected as a subject to receive a specific intervention. However, a candidate subject may be selected to be part of a control group.

According to a third aspect of the invention there is provided a method of selecting a (human) subject for treatment using a therapeutic intervention to maintain, increase, prevent or slow a reduction in renal function, comprising (a) analysing one or more samples obtained from the subject for the level of each of a plurality of biomarkers, (b) comparing the measured levels of each of the plurality of biomarkers with a respective plurality of control levels, (c) determining whether to treat the subject using the therapeutic intervention in dependence on the comparison, wherein the said plurality of biomarkers comprise at least 7 of the group consisting of: (i) adrenomedullin; (ii) alpha-1 antitrypsin; (iii) beta-2-microglobulin; (iv) c16-acylcarnitine; (v) creatine; (vi) creatinine; (vii) fatty acid-binding protein, heart; (viii) fibroblast growth factor 21; (ix) hydroxyproline; (x) kidney injury molecule-1; (xi) N-terminal prohormone of brain natriuretic peptide; (xii) the ratio of symmetric dimethylarginine to asymmetric dimethylarginine; (xiii) symmetric dimethylarginine; and (xiv) uracil. It may be that step (c) also comprises taking into account each of a plurality of clinical covariates pertaining to the subject wherein the clinical covariates comprise at least 5 of the group consisting of: (a) the age of the subject; (b) the gender of the subject; (c) an estimate of glomerular filtration rate (eGFR) of the subject; (d) the level of albumin in urine from the patient; (e) the level of HbA1c in one or more samples obtained from the subject; (f) a measure of the use of ACE inhibitors by the subject; (g) a measure of the use of angiotensin receptor blocker use by the subject.

The therapeutic intervention may comprise a program of administration of a therapeutic entity, for example, a chemical or biological therapeutic entity.

According to a fourth aspect of the invention there is provided a method of monitoring the efficacy of a therapeutic intervention on a (human) subject comprising (a) analysing one or more samples obtained from the subject for the level of each of a plurality of biomarkers, (b) comparing the measured levels of each of the plurality of biomarkers with a respective plurality of control levels, (c) determining risk of decline in renal function in the subject from the comparison; (d) administering the therapeutic intervention to the subject, repeating steps (a), (b) and (c), then (e) comparing the determined risk arising from the comparison of the measured levels before the step of administering the therapeutic intervention to the subject with the determined risk arising from the comparison of the measured levels after the step of administering the therapeutic intervention, and (f) assessing the efficacy of the therapeutic intervention in dependence on that said comparison of the determined risks, wherein the said plurality of biomarkers comprise at least 7 of the group consisting of: (i) adrenomedullin; (ii) alpha-1 antitrypsin; (iii) beta-2-microglobulin; (iv) c16-acylcarnitine; (v) creatine; (vi) creatinine; (XV) cystatin-C; (vii) fatty acid-binding protein, heart; (viii) fibroblast growth factor 21; (ix) hydroxyproline; (x) kidney injury molecule-1; (xi) N-terminal prohormone of brain natriuretic peptide; (xii) the ratio of symmetric dimethylarginine to asymmetric dimethylarginine; (xiii) symmetric dimethylarginine; and (xiv) uracil. It may be that step (c) also comprises taking into account each of a plurality of clinical covariates pertaining to the subject,wherein the clinical covariates comprise at least 5 of the group consisting of: (a) the age of the subject; (b) the gender of the subject; (c) an estimate of glomerular filtration rate (eGFR) of the subject; (d) the level of albumin in urine from the patient; (e) the level of HbA1c in one or more samples obtained from the subject; (f) a measure of the use of ACE inhibitors by the subject; (g) a measure of the use of angiotensin receptor blocker use by the subject.

The therapeutic intervention may for example be to lower blood pressure (e.g. administration of an antihypertensive drug, administration of an ACE inhibitor, an intervention to lower levels of HbA1c, or a treatment to prevent progression of renal disease.

The study may be a clinical trial. The clinical trial may be a clinical trial of a method of treatment, for example administration of a therapeutic entity, such as a chemical or biological entity.

According to a fifth aspect of the invention there is provided a method, carried out by one or more processors of a computer, of processing the levels of a plurality of biomarkers obtained by analysis of one or more samples obtained from the subject, and data concerning the statistical relationship between the levels of said plurality of biomarkers and the risk of renal function decline in the patient, to thereby estimate risk of renal function decline in the subject, wherein the said plurality of biomarkers comprise at least 7 of the group consisting of: (i) adrenomedullin; (ii) alpha-1 antitrypsin; (iii) beta-2-microglobulin; (iv) c16-acylcarnitine; (v) creatine; (vi) creatinine; (vii) fatty acid-binding protein, heart; (viii) fibroblast growth factor 21; (ix) hydroxyproline; (x) kidney injury molecule-1; (xi) N-terminal prohormone of brain natriuretic peptide; (xii) the ratio of symmetric dimethylarginine to asymmetric dimethylarginine; (xiii) symmetric dimethylarginine; and (xiv) uracil.

The method may also comprise processing a plurality of clinical covariates concerning the same subject and the said data may concern the statistical relationship between the levels of said plurality of biomarkers and said plurality of clinical covariates, wherein the clinical covariates comprise at least 5 of the group consisting of: (a) the age of the subject; (b) the gender of the subject; (c) an estimate of glomerular filtration rate (eGFR) of the subject; (d) the level of albumin in urine from the patient; (e) the level of HbA1c in one or more samples obtained from the subject; (f) a measure of the use of ACE inhibitors by the subject; (g) a measure of the use of angiotensin receptor blocker use by the subject.

The data concerning the statistical relationship is typically stored on a tangible, computer readable data storage medium, in electronic communication with the one or more processors. The data may comprise control levels for each of the said plurality of biomarkers. The data may comprise data specifying an algorithm which relates the levels of said plurality of biomarkers, and said plurality of clinical covariates, and the risk of renal function decline in the patient, to the risk of renal function decline in the subject.

The invention also extends in a sixth aspect to a tangible computer readable data storage medium storing program code instructions and data which, when executed by one or more processors of a computer, cause it to carry out the method of the fifth aspect of the invention.

Said tangible computer readable data storage medium may comprise one or more solid stated memories, typically in the form of integrated circuits.

The following features are optional features of each aspect of the invention.

The said plurality of biomarkers may comprise at least 8, at least 10 of, or at least 12 of, or at least 13 of, or may comprise or consist of all of the group consisting of: (i) adrenomedullin; (ii) alpha-1 antitrypsin; (iii) beta-2-microglobulin; (iv) c16-acylcarnitine; (v) creatine; (vi) creatinine; (vii) fatty acid-binding protein, heart; (viii) fibroblast growth factor 21; (ix) hydroxyproline; (x) kidney injury molecule-1; (xi) N-terminal prohormone of brain natriuretic peptide; (xii) the ratio of symmetric dimethylarginine to asymmetric dimethylarginine; (xiii) symmetric dimethylarginine; and (xiv) uracil. It may be that the clinical covariates comprise at least 5 of the group consisting of: (a) the age of the subject; (b) the gender of the subject; (c) an estimate of glomerular filtration rate (eGFR) of the subject; (d) the level of albumin in urine from the patient; (e) the level of HbA1c in one or more samples obtained from the subject; (f) a measure of the use of ACE inhibitors by the subject; (g) a measure of the use of angiotensin receptor blocker use by the subject.

It may be that the said plurality of biomarkers comprises or consists of the group consisting of (x) kidney injury molecule-1, (xii) the ratio of symmetric dimethylarginine to asymmetric dimethylarginine, (iii) beta 2-microglobulin, (ii) alpha-1 antitrypsin, (iv) C-16 acylcarnitine, (viii) fibroblast growth factor-21 (FGF-21) and (xiv) uracil. It may be that the plurality of clinical covariates comprises at least 8, at least 10, or comprising or consisting of all of the group consisting of: (a) the age of the subject; (b) the gender of the subject; (c) an estimate of glomerular filtration rate (eGFR) of the subject; (d) the level of albumin in urine from the patient; (e) the level of HbA1c in one or more samples obtained from the subject; (f) a measure of the use of ACE inhibitors by the subject; (g) a measure of the use of angiotensin receptor blocker use by the subject, (h) a measured of the subject's body mass index, (i) a measure of the duration of the subject's diabetes condition, (j) a measure of the subject's systolic blood pressure, and (k) a measure of the subject's diastolic blood pressure. (c) an estimate of glomerular filtration rate may comprise both an estimate of the subjects' baseline glomerular filtration rate and an average (e.g. weighted average) of their glomerular filtration rate over a period of time. Thus, in the presence of more extensive clinical covariate data these biomarkers may be sufficient to improve prediction.

The said plurality of biomarkers may further comprise (xv) cystatin-C (measured odds ratio 6.34, 95% CI 3.88, 10.97). It may be that the plurality of biomarkers comprises one and only one of cystatin-C and beta-2 microglobulin (measured odds ratio 6.11, 95% CI 3.90, 10.05) as the levels of these biomarkers are tightly correlated (rho=0.86).

It may be that the said plurality of biomarkers includes (vi) creatinine (measured odds ratio 3.43, 95% CI 1.97, 6.36) but the estimate of (c) glomerular filtration rate is also based on a measure of creatinine in a sample obtained from the subject. Surprisingly, we have found that this measurement is not duplicative but improves the accuracy of risk prediction.

It may be that the plurality of biomarkers does not comprise adrenomedullin (measured odds ratio 2.94, 95% CI 2.10, 4.22). It may be that the plurality of biomarkers does not comprise beta-2-microglobulin. We have found that it is possible to generate a panel of 34 biomarkers which is strongly predictive of the risk of rapid decline of renal function not including these biomarkers.

It is possible to use beta-2-microglobulin or cystatin-C as there is a high correlation between levels of these biomarkers.

It may be that the plurality of biomarkers comprises (viii) fibroblast growth factor 21 (measured odds ratio 2.06, 95% CI 1.56, 2.80). This has not previously been identified as a biomarker for renal function decline. Accordingly the invention extends to a method of determining renal function decline risk in a (human) subject, comprising (a) analysing one or more samples obtained from the subject for the level of one or more biomarkers including fibroblast growth factor 21, (b) comparing the measured levels of each of the one or more plurality of biomarkers with a respective one or more control levels, and (c) determining renal function decline risk in the subject from the comparison. An increased level of fibroblast growth factor 21 may be correlated with an increased risk of renal function decline. At least 5, at least 6 or all of the group of clinical covariates consisting of: (a) the age of the subject; (b) the gender of the subject; (c) an estimate of glomerular filtration rate (eGFR) of the subject; (d) the level of albumin in urine from the patient; (e) the level of HbA1c in one or more samples obtained from the subject; (f) a measure of the use of ACE inhibitors by the subject; (g) a measure of the use of angiotensin receptor blocker use by the subject may also be taken into account.

It may be that the plurality of biomarkers comprises (iv) c16-acylcarnitine (measured odds ratio 1.68, 95% CI 1.29, 2.21). This has not previously been identified as a biomarker for renal function decline. Accordingly the invention extends to a method of determining renal function decline risk in a (human) subject, comprising (a) analysing one or more samples obtained from the subject for the level of one or more biomarkers including c16-acylcarnitine, (b) comparing the measured levels of each of the one or more plurality of biomarkers with a respective one or more control levels, and (c) determining renal function decline risk in the subject from the comparison. An increased level of fibroblast growth factor 21 may be correlated with an increased risk of renal function decline. At least 5, at least 6 or all of the group of clinical covariates consisting of: (a) the age of the subject; (b) the gender of the subject; (c) an estimate of glomerular filtration rate (eGFR) of the subject; (d) the level of albumin in urine from the patient; (e) the level of HbA1c in one or more samples obtained from the subject; (f) a measure of the use of ACE inhibitors by the subject; (g) a measure of the use of angiotensin receptor blocker use by the subject may also be taken into account.

It may be that the plurality of biomarkers comprises (ix) hydroxyproline. It is surprising that this is a useful biomarker for renal function decline because in a univariate analysis, it is not statistically significant (measured odds ratio 1.05, 95% CI 0.75, 1.46).

It may be that the plurality of biomarkers comprises (vii) fatty acid-binding protein heart. It is surprising that this is a useful biomarker for renal function decline because in a univariate analysis, it is not statistically significant (measured odds ratio 1.26, 95% CI 0.88, 1.83).

It may be that the plurality of biomarkers comprises (v) creatine. It is surprising that this is a useful biomarker for renal function decline because in a univariate analysis, it is not statistically significant (measured odds ratio 0.63, 95% CI 0.44, 0.89).

It may be that the plurality of biomarkers does not comprise (vii) creatinine. Thus, the said plurality of biomarkers may comprise at least 7, at least 8, at least 10 of, or at least 12 of, or may comprise or consist of all of the group consisting of: (i) adrenomedullin; (ii) alpha-1 antitrypsin; (iii) beta-2-microglobulin or cystatin-C; (iv) c16-acylcarnitine; (v) creatine; (vii) fatty acid-binding protein, heart; (viii) fibroblast growth factor 21; (ix) hydroxyproline; (x) kidney injury molecule-1; (xi) N-terminal prohormone of brain natriuretic peptide; (xii) the ratio of symmetric dimethylarginine to asymmetric dimethylarginine; (xiii) symmetric dimethylarginine; and (xiv) uracil.

The said the plurality of biomarkers may comprise at least 20 of the group consisting of: (ii) alpha-1 antitrypsin; (iv) c16-acylcarnitine; (v) creatine; (vi) creatinine; (vii) fatty acid-binding protein, heart; (viii) fibroblast growth factor 21; (ix) hydroxyproline; (x) kidney injury molecule-1; (xi) N-terminal prohormone of brain natriuretic peptide; (xii) the ratio of symmetric dimethylarginine to asymmetric dimethylarginine; (xiii) symmetric dimethylarginine; (xiv) uracil; (xv) cystatin-C; (xvi) apolipoprotein D or e-selectin; (xvii) fibroblast growth factor 23; (xviii) glutamic acid; (xix) haptoglobin beta-chain; (xx) troponin; (xxi) hypoxanthine; (xxiii) interleukin-2 receptor alpha; (xxiv) latency-associated peptide of transforming growth factor beta 1; (xxv) leucine-rich alpha-2-glycoprotein; (xxvi) lysine; (xxvii) monokine induced by Gamma Inteferon; (xxviii) methylmalonic acid; (xxix) N-acetylaspartate; (xxx) neutrophil gelatinase-associated lipocalin; (xxxi) osteopontin; (xxxii) Tamm-Horsfall urinary glycoprotein; (xxxvii) thymine; (xxxiv) tissue inhibitor of metalloproteinases 1; (xxxvi) tryptophan; (xxxvii) tumour necrosis factor receptor 1; (xxxviii) von Willebrand factor. Hereafter ‘the enlarged group’)

It is possible to use apolipoprotein D or e-selectin because levels of the two are highly correlated (>0.9).

Levels of (xx) Troponin should be measured using a high sensitivity troponin test. Older troponin tests are not sufficiently accurate. However, a variety of sufficiently sensitive tests are commercially available, for example the Elecsys™ Troponin T high sensitive (TnT-hs) test available from Roche.

It may be that the said plurality of biomarkers comprises at least 22, or at least 24, or at least 25, or at least 26, or at least 28, or at least 30, at least 32, at least 33 or may comprise or consist of the said group consisting of: (ii) alpha-1 antitrypsin; (iv) c16-acylcarnitine; (v) creatine; (vi) creatinine; (vii) fatty acid-binding protein, heart; (viii) fibroblast growth factor 21; (ix) hydroxyproline; (x) kidney injury molecule-1; (xi) N-terminal prohormone of brain natriuretic peptide; (xii) the ratio of symmetric dimethylarginine to asymmetric dimethylarginine; (xiii) symmetric dimethylarginine; (xiv) uracil; (xv) cystatin-C; (xvi) apolipoprotein D or e-selectin; (xvii) fibroblast growth factor 23; (xviii) glutamic acid; (xix) haptoglobin beta-chain; (xx) troponin; (xxi) hypoxanthine; (xxiii) interleukin-2 receptor alpha; (xxiv) latency-associated peptide of transforming growth factor beta 1; (xxv) leucine-rich alpha-2-glycoprotein; (xxvi) lysine; (xxvii) monokine induced by Gamma Inteferon; (xxviii) methylmalonic acid; (xxix) N-acetylaspartate; (xxx) neutrophil gelatinase-associated lipocalin; (xxxi) osteopontin; (xxxii) Tamm-Horsfall urinary glycoprotein; thymine; (xxxiv) tissue inhibitor of metalloproteinases 1; (xxxvi) tryptophan; (xxxvii) tumour necrosis factor receptor 1; (xxxviii) von Willebrand factor.

It may be that the plurality of biomarkers comprises (viii) fibroblast growth factor 21. This has not previously been identified as a biomarker for renal function decline.

The level of the ratio of symmetric dimethylarginine to asymmetric dimethylarginine may be expressed as the ratio of measurements of the level of symmetric dimethylarginine and the level of asymmetric dimethylarginine in any suitable units. The level of the (xii) the ratio of symmetric dimethylarginine to asymmetric dimethylarginine and the level of (xiii) symmetric dimethylarginine may be measured by measuring the level of symmetric dimethylarginine and the level of asymmetric dimethylarginine.

The risk may the risk of rapid decline in renal function. Rapid decline in renal function may be characterised by a loss of at least a defined proportion of baseline eGFR over a defined period of time or at a defined rate, for example loss of 40% of baseline eGFR over a maximum period of 3.5 years (equivalent to a loss of at least 11.4% of baseline eGFR per year.:

The determined risk may be a value indicative of a level of risk, for example, a risk level on a suitable scale (e.g. 0-1 or 0-100%). The determined risk may be one of a finite group of risk levels. The determined risk may be a binary value (taking one of two values representative of “at risk” or “not at risk”).

The level of a biomarker may be measured in any appropriate units and may, for example, be an absolute value, or a concentration expressed in units which are proportional to molarity, or proportional to mass per millilitre or proportional to parts per billion, for example. However, the units may for example be in units of the output from an assay, for example a value indicative of the amount of a label for the biomarker, or the size, in any appropriate units, of a peak from a chromatograph. Furthermore, the level of a biomarker may be a level relative to another component of the sample in which the level of biomarker is determined, for example the ratio of the level of the biomarker to the level of a stable isotope of albumin T6.

The level may simply be presence or absence, for example whether an amount or concentration in excess of a detection limit has been detected.

The levels of the biomarkers may be measured in the same sample from a subject, however they may equally be measured in a plurality of different samples from a subject. Typically, the one or more samples are blood samples. Typically the levels of the biomarkers are measured in vitro and/or ex vivo.

The level of a biomarker may be measured by measuring a product derived from the biomarker, for example a cleavage product, such as a tryptic digest, of the biomarker. This approach is especially suitable for liquid chromatography based measurements.

The control levels may be values, or ranges of values. Ranges of values can be expressed as statistical distributions, for example, as an average (e.g. mean) and standard deviation. Levels can be expressed as a number of standard deviations from a mean level, for example.

The risk may the risk of rapid decline in renal function, typically arising from diabetes (for example type II diabetes, and in some embodiments type I diabetes). Rapid decline in renal function may be characterised by: a loss of a defined proportion of baseline eGFR over a defined period of time, or at a defined rate, for example at least 40% of baseline eGFR over a maximum period of 3.5 years (equivalent to a loss of at least 11.4% of baseline eGFR per year.

The level of albumin may be the highest level measured in the subject's urine over a period of time, e.g. the preceding 5 years.

The method may comprise imputing a level of one or more of plurality of biomarkers for which data is missing or where the level is below a detection level and taking the imputed level into account when making the comparison.

For each of the following biomarkers, if the plurality of biomarkers comprises the respective biomarker, an increase level of the respective biomarker may be indicative of an increased risk of renal function decline:

(i) adrenomedullin;

(ii) alpha-1 antitrypsin;

(iii) beta-2-microglobulin;

(iv) c16-acylcarnitine;

(vi) creatinine;

(viii) fibroblast growth factor 21;

(ix) hydroxyproline;

(x) kidney injury molecule-1;

(xi) N-terminal prohormone of brain natriuretic peptide;

(xii) the ratio of symmetric dimethylarginine to asymmetric dimethylarginine;

(xiv) uracil;

(xv) cystatin-C;

(xvi) apolipoprotein D;

(xviii) glutamic acid;

(xx) troponin;

(x) hypoxanthine;

(xxiii) interleukin-2 receptor alpha;

(xxviii) methylmalonic acid;

(xxiv) N-acetylaspartate;

(xxvi) osteopontin;

(xxviii) thymine;

(xxix) tissue inhibitor of metalloproteinases 1;

(xxxi) tumour necrosis factor receptor 1;

(xxxii) von Willebrand factor.

For each of the following biomarkers, if the plurality of biomarkers comprises the respective biomarker, an increase level of the respective biomarker may be indicative of an reduced risk of renal function decline:

(v) creatine;

(xix) haptoglobin beta-chain;

(xxiv) latency-associated peptide of transforming growth factor beta 1;

(xxvi) lysine;

(xxvii) monokine induced by Gamma Inteferon;

(xxvii) Tamm-Horsfall urinary glycoprotein;

(xxx) tryptophan.

(xxxi) E-selectin

For each of the following biomarkers, if the plurality of biomarkers comprises the respective biomarker, an increased level of the respective biomarker may be indicative of an reduced risk of renal function decline, but only when the said clinical covariates are taken into account. When considered individually without the said clinical covariates they would be indicative of an increased risk of renal function decline.

(vii) fatty acid-binding protein, heart;

(xvii) fibroblast growth factor 23;

(xxv) leucine-rich alpha-2-glycoprotein;

(xxv) neutrophil gelatinase-associated lipocalin;

Accordingly, it may be that the plurality of biomarkers comprises (vii) fatty acid-binding protein, heart and an increased level of (vii) fatty acid-binding protein, heart is indicative of a reduced risk of renal function decline. This is surprising because in a univariate analysis, the opposite would be true.

Accordingly, it may be that the plurality of biomarkers comprises (xvii) fibroblast growth factor 23 and an increased level of (xvii) fibroblast growth factor 23 is indicative of a reduced risk of renal function decline. This is surprising because in a univariate analysis, the opposite would be true.

Accordingly, it may be that the plurality of biomarkers comprises (xxv) leucine-rich alpha-2-glycoprotein and an increased level of (xxv) leucine-rich alpha-2-glycoprotein is indicative of a reduced risk of renal function decline. This is surprising because in a univariate analysis, the opposite would be true.

Accordingly, it may be that the plurality of biomarkers comprises (xxv) neutrophil gelatinase-associated lipocalin; and an increased level of (xxv) neutrophil gelatinase-associated lipocalin is indicative of a reduced risk of renal function decline. This is surprising because in a univariate analysis, the opposite would be true.

For (xiii) symmetric dimethylarginine, an increase level of symmetric dimethylarginine is indicative of an reduced risk of renal function decline, but this would not be the case if (xii) the ratio of symmetric dimethylarginine to asymmetric dimethylarginine was not one of the said plurality of biomarkers.

Accordingly, it may be that the plurality of biomarkers comprises both (xiii) symmetric dimethylarginine and (xii) the ratio of symmetric dimethylarginine to assymetric dimethylarginine and an increased level of symmetric dimethylarginine is indicative of a reduced risk of renal function decline and an increased ratio of symmetric dimethylarginine to assymetric dimethylarginine is indicative of an increased risk of renal function decline. It is surprising that improved results are obtained by considering both the level of symmetric dimethylarginine and the ratio of symmetric dimethylarginine to assymetric dimethylarginine.

Adrenomedullin is a 52 amino acid peptide, formed from the activation of a 185 amino acid precursor peptide (preproadrenomedullin). Circulating adrenomedullin consists of both active and glycated inactive forms. Adrenomedullin is a vasodilatory peptide and also has a role in upregulating angiogenesis and protecting cells from oxidative stress.

Alpha-1-Antritrypsin is a 52-kDa serpin protein produced in the liver that blocks the effects of certain enzymes. We measured this biomarker using tryptic digest and MSMS with the fragment that showed the strongest association with renal progression with peptide transition Q1 MS1 of 631.3 and peptide transition Q3 MS2 of 889.5.

Alpha-1-microglobulin is a small 183-amino-acid globular protein that is synthesized by most cells in the body but the liver is the main source. Its primary function is in the degradation of heme and removal of free radicals.

Apolipoprotein D is a member of the lipocalin family. It exists as a homodimer and occurs in the macromolecular complex with lecithin-cholesterol acyltransferase. In plasma, Apolipoprotein D also exists as a disulfide-linked heterodimer with Apolipoprotein A2. Apolipoprotein D is expressed in many tissues but is mainly produced by the brain and testes. It is primarily localized in HDL (60-65%), with most of the remainder in VHDL and only trace amounts in VLDL and LDL.

Aspartic Acid is an acidic a-amino acid with the chemical formula HOOCCH(NH₂)CH₂COOH.

Asymmetric Dimethylarginine (ADMA) and Symmetrical Dimethylarginine (SDMA) and their ratio: During protein turnover three arginine methylated derivatives are released: N(G)-monomethyl-L-arginine (L-NMMA), asymmetrical dimethylarginine (ADMA) and symmetrical dimethylarginine (SDMA). SDMA is largely eliminated by the kidney and several studies have found it to be strongly associated with renal function. Whilst SDMA does not directly inhibit NO synthase there is some evidence that it inhibits the cellular uptake of L-arginine. Also there is some evidence that the protein methyltransferase PRMT 5 that synthesises SDMA regulates interleukin-2-gene expression suggesting that higher levels of SDMA might reflect inflammation. Others have suggested that SDMA levels may be more upregulated than ADMA in states of high protein turnover.

Beta-2-Microglobulin is a component of MHC class I molecules, which are present on all nucleated cells and is shed into the blood, particularly by lymphocytes. Due to its small size it passes through the glomerular membrane of the kidney, but normally less than 1% is excreted in urine due to reabsorption in the proximal tubules of the kidney. Therefore, high plasma levels occur in renal failure, inflammation, as well as some cancers—specifically those associated with B-lymphocytes.

C16 Acylcarnitine, C18 acylcarnitine and C2 acylcarnitine. Acylcarnitines are carnitine esters which play important roles in fatty acid metabolism. The different forms are determined by the chain length profile.

Creatine is a nitrogenous organic acid with the molecular formula C4H9N3O2 that is produced from glycine and arginine. It is principally synthesized by the kidneys and liver before transport to muscle where it is involved in energy production. Low creatine stores may be evidence of a vegetarian diet.

Creatinine is a breakdown product of creatine phosphate. The rate of production is steady and clearance is via the kidneys—principally by filtration at the glomerulus with no active reabsorption. Serum creatinine is used as a routine measure of renal function as changes in the concentration principally reflect changes in kidney filtration. Serum creatinine is used in many of the equations established for calculating eGFR such as the MDRD4 (used in this study) and CKD-EPI equations.

Cystatin-C is a 13 kDa protein that is a potent inhibitor of the C1 family of cysteine proteases. It is normally expressed in vascular wall smooth muscle cells. It is cleared by filtration at the kidney glomerulus and is an established marker of glomerular filtration.

E-Selectin is one of a family of cell adhesion molecules. E-Selectin is expressed only on cytokine activated endothelial cells. Soluble E-Selectin arises from proteolytic cleavage of the surface-expressed molecule. It plays an important role in inflammation including recruitment of leukocytes to areas of damage.

Fatty Acid Binding Protein, Heart is a member of the fatty acid binding protein family and is involved in fatty acid metabolism acting as a transporter for fatty acids to the mitochondria. Fatty acid binding protein heart is a 15 kDa cytoplasmic protein present predominantly in myocardial cells. It is a marker of acute myocardial infarction and may also be a useful marker of cardiovascular risk.

Fibroblast Growth Factor 21 is a member of the fibroblast growth factor family. It is a polypeptide of 181 amino acids secreted predominantly by the liver and adipose tissue. Fibroblast Growth Factor 21has been shown to play an important role in lipid and energy metabolism. Fibroblast Growth Factor 21 stimulates glucose uptake in differentiated adipocytes via the induction of glucose transporter SLC2A1/GLUT1 expression. Several previous studies have reported cross sectional associations of FGF 21 with eGFR and also with albuminuria.

Fibroblast Growth Factor 23 is a member of the fibroblast growth factor family. It is produced by osteocytes and osteoblasts in response to high circulating phosphate levels or elevated parathyroid hormone. It promotes decreased renal resorption of phosphate by downregulating phosphate transporters and by suppressing vitamin D production. It also decreases the intestinal absorption of phosphate. In human chronic kidney disease, plasma Fibroblast Growth Factor 23 levels increase during early stages of kidney malfunction.

Glutamic acid is a non-essential proteinogenic amino acid. It can be used along with glutamine as a marker of good sample storage as samples stored without proper freezing will show a rise in the conversion of glutamate to glutamic acid.

Growth differentiation factor 15 is a protein belonging to the transforming growth factor beta superfamily that ha a role in regulating inflammatory and apoptotic pathways in injured tissues and during disease processes.

Haptoglobin Beta Chain is a protein chain which in combination with the haptoglobin alpha chain makes up the protein haptoglobin which binds free haemoglobin released from erythrocytes.

Hydroxyproline is an amino acid produced by the hydroxylation of proline in the endoplasmic reticulum. It is a major component of the protein collagen and along with proline plays a key role in collagen stability.

Hypoxanthine is a purine derivative with the molecular formula C₅H₄N₄O. Hypoxanthine is released into the circulation from muscle after the consumption of ATP and thus serum levels have been shown to rise following exercise or ischaemia. Interleukin-2 receptor alpha is one of the three proteins chains that make up the Interleukin-2 receptor, the others being Interleukin-2 Beta and Interleukin-2 Gamma. The soluble form results from extracellular proteolysis and blood levels correlate with increased T and B cell activation and immune system activation.

Kidney Injury Molecule 1 is an immunoglobulin superfamily cell-surface protein expressed on the apical membrane of proximal tubule cells and is highly upregulated on the surface of damaged kidney epithelial cells. Urinary concentrations of Kidney Injury Molecule 1 have been shown to rise in response to acute renal injury. Urinary KIM-1 has been evaluated as a prognostic marker in diabetic kidney disease but has not been consistently found to be a strong independent predictor of progression. In this study we have measured Kidney Injury Molecule 1 in serum.

Latency associated peptide of transforming growth factor 1 beta is encoded by the transforming growth factor 1 beta gene and co-secreted with transforming growth factor 1 beta. Cleavage of the two proteins gives rise to both Latency associated peptide of transforming growth factor 1 beta and the mature transforming growth factor 1 beta itself.

Leucine-rich alpha-2-glycoprotein is a secretory type 1 acute phase protein that belongs to the leucine-rich repeat family of proteins, which have been shown to be involved in protein-protein interaction, signal transduction, and cell adhesion and development. Leucine-rich alpha-2-glycoprotein is expressed during granulocyte differentiation. It binds strongly to Cytochrome C, suggesting it may be involved in apoptosis.

Lysine is an essential amino acid with the chemical formula HO₂CCH(NH₂)(CH₂)₄NH₂.

Methylmalonic acid (MMA) is a dicarboxylic acid that is a C-methylated derivative of malonate. Higher levels are found in the presence of vitamin B12 deficiency.

Monokine Induced by Gamma Interferon is a small cytokine belonging to the family of CXC chemokines. It is a T-cell chemoattractant whose expression is induced by interferon gamma. It plays a role in both immune and inflammatory responses and is chemotactic for activated T-cells.

N-acetylaspartate is a small amino acid with a formula of C6H9NO5 synthesized primarily by neuronal mitochondria from the amino acid aspartic acid and acetyl-coenzyme A by the enzyme aspartate-N-acetyltransferase. It has been demonstrated to be released into the circulation following neuronal injury such as stroke and other neuronal disorders.

N-terminal prohormone of brain natriuretic peptide (NT-ProBNP) is a 76 amino acid N-terminal inactive protein that is cleaved from pro-BNP to release brain natriuretic peptide (BNP). Blood levels of NT-proBNP and the active peptide BNP are used for screening and diagnosis of acute congestive heart failure. The plasma concentrations of both BNP and NT pro-BNP are also typically increased in patients with asymptomatic or symptomatic left ventricular dysfunction.

Neutrophil Gelatinase-Associated Lipocalin (NGAL) is a protein of the lipocalin family. It forms a disulfide bond-linked heterodimer with MMP-9. It mediates an innate immune response to bacterial infection by sequestrating iron. It is expressed principally in neutrophils, but is also expressed in low levels in kidney along with other tissues. NGAL levels rise in urine and blood within 2 hours of renal insult making NGAL a biomarker for acute renal injury.

Osteopontin consists of 314 amino acids and acts as a cytokine synthesized by the kidney and is involved in enhancing the production of interferon-gamma and IL-12 and reducing the production of IL-10. Osteopontin is essential in the pathway that leads to type I immunity. It appears to form an integral part of the mineralized matrix. Sialic acid is a term for a group of derivatives of the monosaccharide neuraminic acid, of which the most abundant in humans is N-acetylneuraminic acid. Total sialic acid has been found to be elevated in serum in the presence of renal disease.

Tamm-Horsfall protein, also known as uromodulin, is a monomeric glycoprotein of ^{˜}85 kDa with ^{˜}30% carbohydrate moiety that is heavily glycosylated. It is the most abundant protein present in the urine of healthy subjects. It is produced in the kidney and secreted into the urine by the thick ascending limb of the Loop of Henley. Recent studies have found that Tamm-Horsfall protein is present in serum and is associated with the presence of renal disease.

Thrombomodulin, also known as CD141, is an integral membrane protein expressed on the surface of endothelial cells. It functions as a cofactor in the thrombin-induced activation of protein C by forming a 1:1 complex with thrombin. This complex then stimulates fibrinolysis.

Thymine is a nucleic acid with the molecular formula C₅H₆N₂O₂. It is present in DNA and can be derived by the methylation of uracil.

Tissue Inhibitor of Metalloproteinases 1 (TIMP-1) is a member of a family of secreted proteolytic enzymes that are involved in the biosynthesis of connective tissue via inhibiting actions of matrix metalloproteinases TIMP-1 is a glycoprotein and a major regulator of extracellular matrix synthesis and degradation.

Trefoil Factor 3 also known as intestinal trefoil factor is a small peptide that, as with other Trefoil Factor peptides, is highly expressed in tissues containing mucus-producing cells. It exists both in a 60 amino-acid monomeric form and a 118 amino acid dimeric form. It is thought to have a role in epithelial repair. More recently it has been shown to be expressed in the renal medulla and more specifically to tubular cells in the renal cortex. TFF3 has been shown to be higher in serum and urine of those with lower eGFR in the general population.

Troponin T is a myofibrillar protein which along with Troponin I and Troponin C makes up the troponin complex that is a key component of muscle contractility. Cardiac Troponin T is a recognised biomarker for acute cardiac injury but more recently high sensitivity assays have shown that plasma and serum troponin T measures are also biomarkers of cardiovascular risk.

Tryptophan is an essential amino acid with the molecular formula C₁₁H₁₂N₂O₂. It is required for the production of serotonin and niacin.

Tumor Necrosis Factor Receptor I and Tumor necrosis factor receptor 2 are the soluble forms of the TNF receptor. Two types of soluble TNF receptors have been identified in human serum and urine that neutralize the biological activities of TNF-alpha and TNF-beta. These binding proteins represent truncated forms of the two types of high-affinity cell surface receptors for TNF (TNFR-p60 Type B and TNFR-p80 Type A). Soluble Tumor Necrosis Factor Receptor I corresponds to TNFR-p60 Type B. Soluble Tumor Necrosis Factor Receptor 2 corresponds to TNFR-p80 Type A. In the TNF superfamily nomenclature, TNFR1 and TNFR2 are referred to as TNFRSF1A and TNFRSF1B, respectively. These apparent soluble forms of the receptors appear to arise as a result of shedding of the extracellular domains of the membrane-bound receptors.

Uracil is a pyramidine derivative with the molecular formula of C₄H₄N₂O₂. It is one of the nucleic acids in RNA. It acts as a cofactor in the production of many enzymes.

Vascular Cell Adhesion Molecule-1 (VCAM-1) is made up of multiple immunoglobulin domains and is expressed in large and small blood vessels after stimulation by cytokines. It supports the adhesion of lymphocytes, monocytes, natural killer cells, eosinophils, and basophils through its interaction with leukocyte very late antigen-4 (VLA-4). The VCAM-1/VLA4 interaction mediates firm adherence of circulating non-neutrophilic leukocytes to endothelium and may play a pathophysiologic role both in immune responses and in leukocyte emigration to sites of inflammation.

von Willebrand Factor (vWF), is a large multimeric glycoprotein involved in the maintenance of hemostasis. The basic monomer of vWF is a 2050amino acid protein. In the plasma, vWF exists as a heterogenous population of large polymers to which the coagulant factor VIII is complexed by non-covalent bonds. It promotes adhesion of platelets to the sites of vascular injury by forming a molecular bridge between sub-endothelial collagen matrix and platelet-surface receptor complex GPIb-IX-V. It also acts as a chaperone for coagulation factor VIII, delivering it to the site of injury, stabilizing its heterodimeric structure and protecting it from premature clearance from plasma. vWF is synthesized in endothelial cells and in megakaryocytes as a number of subunits that polymerize and combine with the factor VIII to form a large complex. It is an acute-phase reactant.

### BRIEF DESCRIPTION OF THE DRAWINGS

An example embodiment of the invention will now be described with reference to the following Figures in which:

FIG. 1 is a flow chart of a method for calculating a risk of rapid decline of renal function according to the invention;

FIG. 2 is a schematic diagram of a computer programmed to calculate a risk of rapid decline of renal function according to the invention;

FIG. 3 is a graph of Area under the Receive Operating Characteristic (AUROC) for the models based on the number of biomarkers;

FIG. 4 is a graph of Area under the Receive Operating Characteristic (AUROC) for a model based on clinical covariates alone and for models using spare and extended biomarker panels;

FIG. 5 is a volcano plot showing association of individual biomarkers and clinical covariates by odds ratio for association with rapid progression of eGFR adjusted for clinical covariates when examined singly. The labelled points are where there was a level of significance −log 10(pvalue)>9 or a fold-change greater than +/−>0.6;

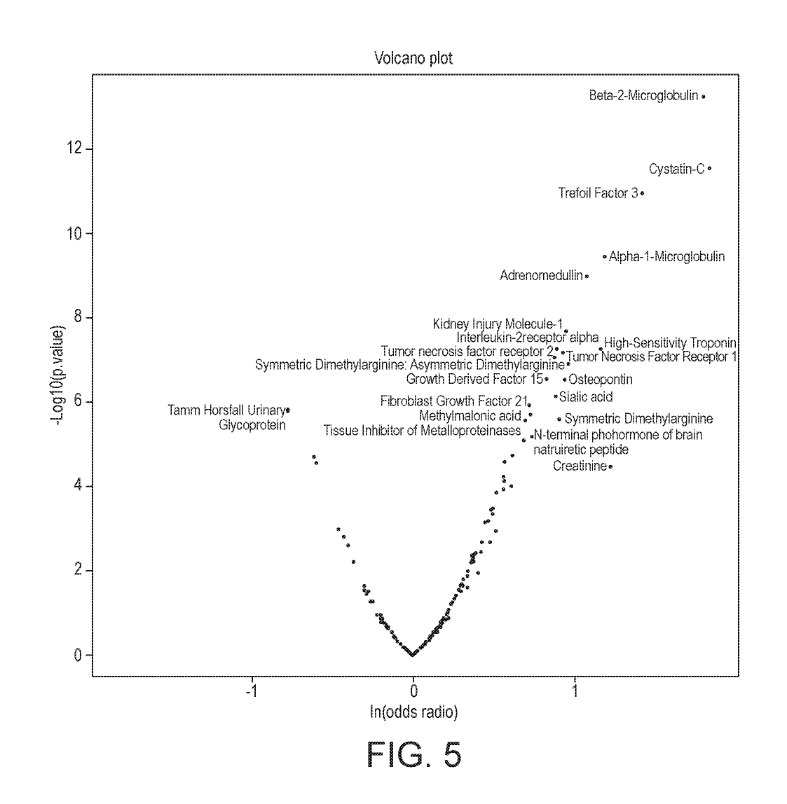

FIG. 6 shows performance metrics of models by number of biomarkers retained and selection method. Model performance plots showing the AUROC achieved with the forward selection panel (shown by filled square) compared with the performance of the top down selected panels (shown by open diamonds) with the number of retained biomarkers in the top down selection allowed to vary up to 35 biomarkers. (FIG. 6A) On top of the AUROC achieved by age, sex, HbA1c, albuminuria, eGFR. ACE Inhibitor and Angiotensin Receptor Blocker use alone (FIG. 6B) On top of the AUROC achieved by an extended set of clinical covariates including longitudinal eGFR (see methods for full list);

FIG. 7 shows performance of panels of biomarkers chosen by forward selection and top down selection compared with clinical data alone. Performance plots for the best overall and sparse biomarker models including clinical covariates age, sex, HbA1c, albuminuria, eGFR and ACE Inhibitor and Angiotensin Receptor Blocker use (line 200), clinical covariates and forward selection biomarkers (line 201) and clinical covariates and 35 biomarker panel (line 202) (FIG. 7A) Area under the ROC curves FIG. 7B) positive predictive value plot;

### DETAILED DESCRIPTION

### FIRST EXAMPLE

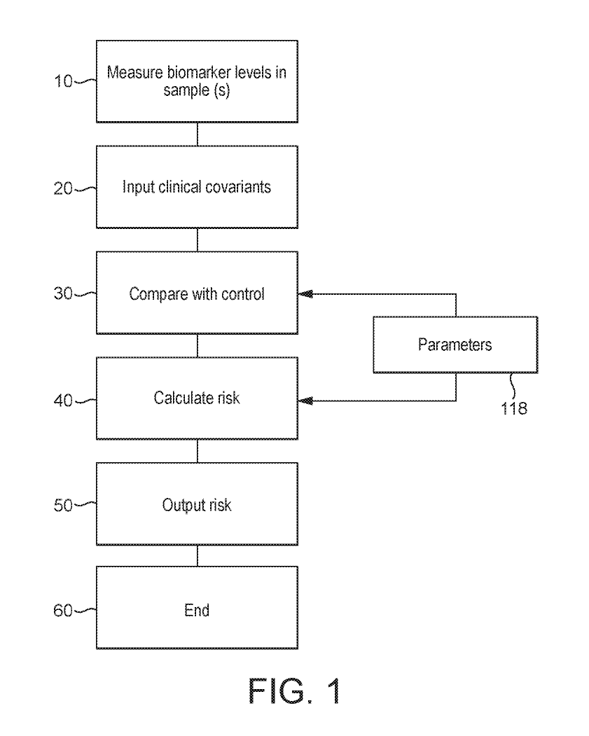

With reference to FIG. 1, in order to assess the risk of a subject suffering from a rapid decline in renal function in the future, for example arising from type II diabetes, blood samples are taken from the subject and assayed 10 for the presence of the biomarkers set out in Table 1 below.

TABLE 1-14
biomarker panel

| | Measurement | |
|---|---|---|
| Biomarker | Technique | Notes |
| Adrenomedullin | Microsphere assay | |
| Alpha-1 antitrypsin | ESI-MSMS | Measure tryptic digest and |
| | | given as a ratio to a stable |
| | | isotope of albumin T31 added |
| | | during the measurement |
| | | process |
| Beta-2- | Microsphere assay | |
| microglobulin | | |
| C16-acylcarnitine | ESI-MSMS | |
| Creatine | ESI-MSMS | |
| Creatinine | ESI-MSMS | |
| Fatty acid-binding | Microsphere assay | |
| protein, heart | | |
| Fibroblast Growth | Microsphere assay | |
| Factor 21 | | |
| Hydroxyproline | ESI-MSMS | |
| Kidney Injury | Microsphere assay | |
| Molecule-1 | | |
| N-terminal | Microsphere assay | |
| Prohormone of | | |
| Brain Natriuretic | | |
| Peptide | | |
| The ratio of | ESI-MSMS | |
| Symmetric | | |
| dimethylarginine to | | |
| Asymmetric | | |
| dimethylarginine | | |
| Symmetric | ESI-MSMS | |
| dimethylarginine | | |
| Uracil | ESI-MSMS | |

The levels of the biomarkers can be measured in a variety of different ways known to persons skilled in the art.

Typically, individual biomarkers can be measured using a specific recognition element, such as an antibody, antibody fragment, chemical entity etc. which binds specifically to the biomarker, and a label, or other detection technology. For example, the levels of individual biomarkers may be measured using enzyme linked immunosorbent assays (ELISAs) or other immunoassays (e.g. ECLIA, electroluminescent immunoassay) incorporating antibodies (or antibody fragments, or analogues or derivatives thereof) as specific recognition elements for the respective biomarker, and a label to facilitate detection. Biomarker levels can be measured for multiple biomarkers in parallel with multiplexed microsphere based detection assays using the xMAP multiplexing assaying technology, available from Luminex Corporation (Austin, Tex., UK) (xMAP is a trade mark of Luminex Corporation). This is referred to as “Microsphere assay” in Table 1. Biomarkers can also be measured using chromatographic techniques, for example a liquid chromatography-mass spectrometry (LS-MS) technique, such as liquid chromatography-electrospray ionization-tandem mass spectrometry (ESI-MSMS in Table 1) in which biomarkers are separated using suitably configured liquid chromatography apparatus and detected using mass spectrometry. These methods are described further in the experimental section below. Some biomarkers may be pre-treated, for example digested with trypsin, prior to analysis and in this example this approach is applied for alpha-1 antitrypsin. This is especially helpful with chromatography based techniques such as LS-MS and generally leads to multiple peaks being detected, some or all of which may be measured to determine the level of the corresponding biomarker. For biomarkers identified by tryptic digest in this way, rather than measuring an absolute concentration a semiquantitative measure is derived using the ratio of the concentration of the biomarker to a stable isotope of albumin T6 added during sample preparation.

In addition, the following clinical covariates are recorded 20 for each subject: their age; their gender; the level of glycated haemoglobin (HbA1c) in a blood sample using Diabetes Control and Complications Trial (DCCT) aligned assays; the level of albumin in their urine grouped into normoalbuminuria (ie below the threshold for microalbuminuria relevant to the assay used such as microalbumin concentration, 24 hour protein concentration, urinary Albumin:Creatinine Ratio etc) or albuminuria which included micro or macroalbuminuria (again, as defined for the appropriate assay); their estimated glomerular filtration rate (eGFR) determined from a blood test for creatinine using the Modification of Diet in Renal Disease 4 (MDRD4) equation: eGFR=186×(creatinine in mmol/l/88.4)−1.154×(age−0.203)×0.742 (if female)×(1.210 if black), and whether (and optionally to what extent) they use ACE (angiotensin-converting-enzyme) inhibitors and Angiotensin Receptor Blockers.

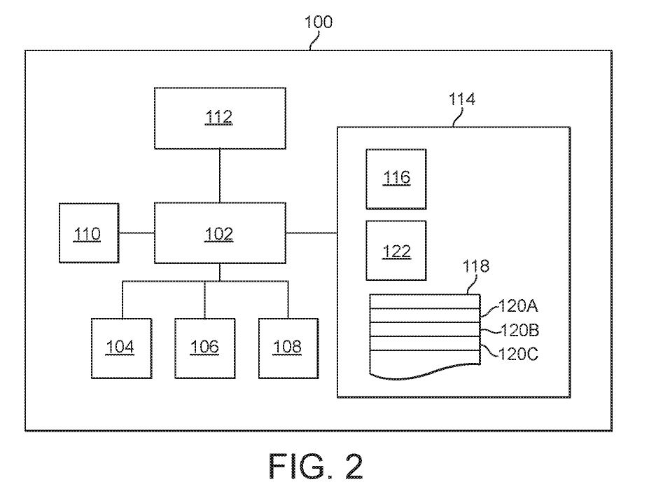

With reference to FIG. 2, this data is entered into a computer 100, either manually using a data input peripheral, such as a keyboard 104, mouse 106 or touch interface 108, or automatically, directly from measurement apparatus through a network interface 110 (such as an Ethernet interface). The computer has one or more microprocessors 102 in electronic communication with memory 114 which stores a computer program 116 to calculate the risk of rapid decline of renal function, parameters 118 including parameters relating to each of a plurality of biomarkers 120A, 120B, 120C, which are required by the computer program, and the received data concerning a subject 122. The computer calculates 40 the risk of an individual subject suffering from rapid decline of renal function by the following procedure:

Data for all those assessed is Gaussianized to maximise the prediction of the model and all clinical covariate and biomarker data. An estimated outcome (which reflects the rate of change of eGFR for a patient) as a continuous measure from a linear regression model that includes all clinical covariates and biomarker data, as set out below. The generated outcome is an indicator of rate of eGFR decline for that individual with those having larger scores being those at higher risk than those with lower scores. It is also possible to use the coefficients generated from our case-control model but models built from other populations will be more effective if coefficients are derived from their own population data. The threshold for selection depends on the number of individuals needing to be identified or a predetermined cut-point.

In order to estimate the outcome, any missing biomarker measurements and environmental and clinical covariates are next imputed by using the imputation model pre-trained on the training data as described below (see “Statistical Methods”).

The quantitative risk score is estimated by the conditional probability p(yᵢ=1|xᵢ) of sample i being the case for rapid decline in renal function, given the set of the imputed biomarkers and environmental and/or clinical covariates xᵢ, by using the logistic regression model p(yᵢ=1|xᵢ)=1/(1+exp(−w₀−w^{T}xᵢ)). Model parameters w₀, w are estimated from the training data by using the novel modifications of the forward-selection or LASSO procedures (see “Statistical Methods”). The modifications include the use of the novel imputation method accounting for the mixture of random and systematic causes for missingness (“Data Cleaning and Imputation”), and the nested filtering procedure accounting for over-shrinking and redundancy for the high-dimensional prediction of the extreme cases (rapid progressors as opposed to case/control status for diabetic nephropathy)—see “Methodology for Addressing Bottlenecks in Biomarker Identification below.

The length of vector w of covariate weights (model parameters) equals to the number of biomarkers and clinical/environmental covariates (one coefficient per covariate). The vector is sparse, that is many of the elements are equal to zero. Zeros correspond to the discarded covariates that are not included in the resulting biomarker panels; non-zero entries of the vector w correspond to biomarkers retained in the resulting biomarker panel. The signs and magnitudes of the weights indicate the directions and sizes of the effects of the corresponding biomarkers and clinical covariates from the resulting panel on the risk of rapid progression of renal decline.

The probabilities estimated according to (2) constitute patients' individual-level quantitative risk scores and are to be used by trained physicians or clinical trialists to determine a preventive intervention, segment clinical trials, or evaluate efficacy of ongoing therapy.

For each biomarker the following data is employed.

TABLE 2

| | | 95% Confidence | | |
|---|---|---|---|---|
| Biomarker | Odds ratio | Interval | | p-value |
| Adrenomedullin | 1.07 | 0.56, | 2.04 | 0.8370 |
| Alpha-1 antitrypsin | 2.05 | 1.38, | 3.14 | 0.0111 |
| Beta-2- | 3.19 | 1.56, | 6.84 | 0.0019 |
| microglobulin | | | | |
| C16-acylcarnitine | 1.76 | 1.16, | 2.73 | 0.0090 |
| Creatine | 0.65 | 0.41, | 1.01 | 0.0590 |
| Creatinine | 3.52 | 1.54, | 8.76 | 0.0042 |
| Fatty acid-binding | 0.63 | 0.38, | 1.02 | 0.0588 |
| protein, heart | | | | |
| Fibroblast Growth | 1.69 | 1.06, | 2.75 | 0.0288 |
| Factor 21 | | | | |
| Hydroxyproline | 1.73 | 1.12, | 2.72 | 0.0151 |
| Kidney Injury | 1.93 | 1.18, | 3.27 | 0.0111 |
| Molecule-1 | | | | |
| N-terminal | 1.84 | 1.15, | 3.01 | 0.0123 |
| Prohormone of | | | | |
| Brain Natriuretic | | | | |
| Peptide | | | | |
| The ratio of | 8.36 | 3.83, | 20.40 | <0.0001 |
| Symmetric | | | | |
| dimethylarginine to | | | | |
| Asymmetric | | | | |
| dimethylarginine | | | | |
| Symmetric | 0.32 | 0.13, | 0.72 | 0.0075 |
| dimethylarginine | | | | |
| Uracil | 1.84 | 1.22, | 2.84 | 0.0046 |

The computer outputs 50 a risk for a patient. In some embodiments, the computer calculates a risk of the subject being at risk of rapid decline of renal function. In other embodiments, the computer allocates the subject to one or a group of risk categories, which may be as simple as “at increased risk” or “not at increased risk”.

The resulting data can be used in a number of ways. For example, the data can be used as part of a procedure for selecting subjects for a clinical trial. In this case, subjects who are found to be at increased risk are selected as candidates for a clinical trial to assess the efficacy of an intervention, such as administration of a drug. Candidate subjects are typically then screened against additional criteria and may then be randomly distributed between control and experimental groups. As part of the clinical trial, the selected subjects may have one or more measures of kidney function, such as albuminuria or eGFR measured repetitively. They may be categorised into different stages of kidney disease. The change in these measurement or stages through the trial may be monitored.

By selecting patients who are found to have been at increased risk by the method of the present invention, the clinical trial may need to recruit fewer subjects, or may be completed more quickly than would otherwise be the case.

The invention can then be used to assess the progress of a course of treatment in a subject. Their risk of suffering rapid decline of renal function may be determined by the procedure described above at different times, before, during or after a course of treatment, and whether the course of treatment is working can be determined from the calculated risk.

The invention is also useful to assess whether a subject should be treated with a course of treatment. They may be treated with a course of treatment if the calculation determines that their risk of suffering rapid decline of renal function exceeds a threshold, of it they are allocated to a risk category indicative of increased risk of rapid decline of renal function.

### SECOND EXAMPLE

In a second example, an expanded panel of biomarkers is instead employed, as set out in Table 3.

TABLE 3
Expanded biomarker panel

| | Measurement | |
|---|---|---|
| Biomarker | Technique | Notes |
| Alpha-1 Antitrypsin | ESI-MSMS | Measured by tryptic |
| | | digest and expressed as |
| | | ratio between biomarker |
| | | and concentration of |
| | | added stable isotope of |
| | | albumin T6 |
| Apolipoprotein D | Microsphere | |
| | Assay | |
| C16-acylcarnitine | ESI-MSMS | |
| Creatine | ESI-MSMS | |
| Creatinine | ESI-MSMS | |
| Cystatin-C | Microsphere | |
| | Assay | |
| Fatty Acid-Binding Protein | Microsphere | |
| heart | Assay | |
| Fibroblast Growth Factor 21 | Microsphere | |
| | Assay | |
| Fibroblast growth factor 23 | Microsphere | |
| | Assay | |
| Glutamic acid | ESI-MSMS | |
| Haptoglobin beta-chain | ESI-MSMS | Measured by tryptic |
| | | digest and expressed as |
| | | ratio between biomarker |
| | | and concentration of |
| | | added stable isotope of |
| | | albumin T6 |
| Hydroxyproline | ESI-MSMS | |
| Hypoxanthine | ESI-MSMS | |
| Interleukin-2 receptor alpha | Microsphere | |
| | Assay | |
| Kidney Injury Molecule-1 | Microsphere | |
| | Assay | |
| Latency-Associated Peptide of | Microsphere | |
| Transforming Growth Factor | Assay | |
| beta 1 | | |
| Leucine-rich alpha-2- | ESI-MSMS | Measured by tryptic |
| glycoprotein | | digest and expressed as |
| | | ratio between biomarker |
| | | and concentration of |
| | | added stable isotope of |
| | | albumin T6 |
| Lysine | ESI-MSMS | |
| Methylmalonic acid | ESI-MSMS | |
| Monokine Induced by Gamma | Microsphere | |
| Interferon | Assay | |
| N-acetylaspartate | ESI-MSMS | |
| Neutrophil Gelatinase- | Microsphere | |
| Associated Lipocalin | Assay | |
| N-terminal prohormone of | Microsphere | |
| brain natriuretic peptide | Assay | |
| Osteopontin | Microsphere | |
| | Assay | |
| Symmetric Dimethylarginine | ESI-MSMS | |
| Symmetric Dimethylarginine: | ESI-MSMS | |
| Asymmetric Dimethylarginine | | |
| Tamm-Horsfall Urinary | Microsphere | |
| Glycoprotein | Assay | |
| Thymine | ESI-MSMS | |
| Tissue Inhibitor of | Microsphere | |
| Metalloproteinases 1 | Assay | |
| Troponin | ELISA | Elecsys troponin T |
| | | high sensitivity assay |
| | | (Roche Diagnostics) |
| Tryptophan | ESI-MSMS | |
| Tumor Necrosis Factor | Microsphere | |
| Receptor I | Assay | |
| Uracil | ESI-MSMS | |
| von Willebrand Factor | Microsphere | |
| | Assay | |

The ratio of measured level to measured level of stable isotope of albumin T6 can be established by measuring the level of the biomarker and the level of added stable isotope of albumin T6 in the same sample and calculating the ratio of the amount present.

The calculation is repeated as before, except that it incorporates each of the measured biomarkers included in the table below:

TABLE 4

| | | 95% | | |
|---|---|---|---|---|
| | Odds | Confidence | | |
| Biomarker | Ratio* | Interval | | p-value |
| Alpha-1 Antitrypsin | 3.77 | 1.91, | 8.17 | 0.0003 |
| Apolipoprotein D | 1.94 | 1.07, | 3.70 | 0.0345 |
| C16-acylcarnitine | 2.50 | 1.37, | 4.82 | 0.0041 |
| Creatine | 0.48 | 0.21, | 1.00 | 0.0593 |
| Creatinine | 7.65 | 2.35, | 29.52 | 0.0015 |
| Cystatin-C | 4.29 | 1.51, | 13.84 | 0.0093 |
| Fatty Acid-Binding Protein heart | 0.32 | 0.14, | 0.67 | 0.0040 |
| Fibroblast Growth Factor 21 | 2.54 | 1.24, | 5.67 | 0.0155 |
| Fibroblast growth factor 23 | 0.27 | 0.11, | 0.58 | 0.0015 |
| Glutamic acid | 1.67 | 0.83, | 3.46 | 0.1530 |
| Haptoglobin beta-chain | 0.36 | 0.16, | 0.71 | 0.0062 |
| Hydroxyproline | 2.02 | 1.10, | 3.92 | 0.0280 |
| Hypoxanthine | 1.34 | 0.75, | 2.50 | 0.3340 |
| Interleukin-2 receptor alpha | 2.81 | 1.21, | 7.00 | 0.0197 |
| Kidney Injury Molecule-1 | 2.98 | 1.42, | 6.79 | 0.0057 |
| Latency-Associated Peptide of | 0.67 | 0.34, | 1.25 | 0.2230 |
| Transforming Growth Factor | | | | |
| beta 1 | | | | |
| Leucine-rich alpha-2-glycoprotein | 0.45 | 0.20, | 0.93 | 0.0388 |
| Lysine | 0.80 | 0.35, | 1.80 | 0.5940 |
| Methylmalonic acid | 1.38 | 0.75, | 2.63 | 0.3130 |
| Monokine Induced by Gamma | 0.45 | 0.20, | 0.97 | 0.0461 |
| Interferon | | | | |
| N-acetylaspartate | 1.42 | 0.70, | 2.99 | 0.3430 |
| Neutrophil Gelatinase-Associated | 0.44 | 0.21, | 0.86 | 0.0203 |
| Lipocalin | | | | |
| N-terminal prohormone of brain | 2.68 | 1.32, | 5.87 | 0.0088 |
| natriuretic peptide | | | | |
| Osteopontin | 1.46 | 0.65, | 3.48 | 0.3690 |
| Symmetric Dimethylarginine | 0.12 | 0.03, | 0.44 | 0.0022 |
| Symmetric Dimethylarginine: | 35.74 | 9.26, | 189.02 | 2.57E−06 |
| Asymmetric Dimethylarginine | | | | |
| Tamm-Horsfall Urinary | 0.85 | 0.44, | 1.67 | 0.6250 |
| Glycoprotein | | | | |
| Thymine | 1.89 | 1.03, | 3.68 | 0.0483 |
| Tissue Inhibitor of | 1.83 | 0.84, | 4.14 | 0.1310 |
| Metalloproteinases 1 | | | | |
| Troponin | 2.48 | 1.05, | 6.37 | 0.0470 |
| Tryptophan | 0.60 | 0.29, | 1.17 | 0.1410 |
| Tumor Necrosis Factor | 3.01 | 1.49, | 6.47 | 0.0029 |
| Receptor I | | | | |
| Uracil | 1.27 | 0.70, | 2.31 | 0.4320 |
| von Willebrand Factor | 1.15 | 0.62, | 2.11 | 0.6600 |

The invention provides an improved level of accuracy in detecting individuals at a high risk of rapid decline in renal function. Advantageously, most or all of the biomarkers can be measured using just two assays, one chromatographic assay (which can distinguish between a plurality of biomarkers) and one multiplexed immunoassay using differently labelled specific recognition elements (such as antibodies, antibody fragments or analogues thereof) for different biomarkers. One of the above listed biomarkers, Troponin T, was not measurable by either of these methods and instead was measured by ELISA as detailed in table 5.

Experimental Methods

The biomarkers panels were selected and their correlation with rapid decline in renal function was determined by the following methods.

153 cases of rapid progression of renal disease were identified from the Go-DARTS cohort in Scotland along with 154 controls. A panel of 207 biomarkers was measured in baseline samples to identify biomarkers that might improve prediction of renal decline.

Go-DARTS (Genetics of Diabetes Audit and Research Tayside Study) is a cohort of individuals registered with type 2 diabetes were individuals have both a biosamples and clinical data collected at enrolment along with longitudinal diabetes related measures collected via data linkage to the subjects electronic health record providing retrospective and prospective data of routine clinical measures as well as data on prescribing and in-patient admissions. Patients were enrolled in the study between December 1998 and May 2009 and are continuously followed up to the present. All those attending participating clinics (which cover all of the health Board area of Tayside, Scotland) were invited to participate. The final sample comprises approximately 75% of all those with type 2 diabetes residing in the area. Diabetes status is based on a clinical record of a diagnosis of diabetes and has been validated against clinical record data and on-going prescription and biochemistry laboratory data. Patients gave a blood sample at study entry and agreed to have their routine and diabetes specific clinical and mortality records ascertained prospectively. Covariate data including prescription information, blood pressure and anthropometry and biochemical test results are obtained by extraction from the ongoing primary care records and hospital diabetes electronic record (SCI-DC).

The phenotype for this study was designed around the typical enrolment criteria for assessing reno-protective drugs. Samples were selected from individuals with CKD3 (i.e. an eGFR of 30-60 ml/min/1.73 m2) on the day of study enrolment. EGFR was calculated from a single serum creatinine measured at a clinical laboratory on the day of enrolment into Go-Darts by the MDRD4 equation (Levey A S, Coresh J, Greene T, Stevens L A, Zhang Y L, Hendriksen S, et al. Using standardized serum creatinine values in the modification of diet in renal disease study equation for estimating glomerular filtration rate. Ann Intern Med. 2006; 145(4): 247-54). All cases had at least one subsequent calculated eGFR in their clinical record that was <60% of their baseline eGFR within a maximum follow-up of 3.5 years. In contrast, all controls were followed up for >3.5 years, never had an eGFR<80% of their baseline value and at their most recent follow up had an eGFR>95% of the baseline value. Any individual without exposure to anti-hypertensive treatments within a year of their baseline eGFR measurement was excluded. For cases individuals with a hospital admission for Acute Renal Failure following enrolment in the study were also excluded. The study included 307 samples from 153 cases and 154 controls.

A set of biomarkers was selected to measure in the study that was based upon three principal factors: 1) prior evidence for an association with nephropathy, 2) existence of a reliable assay that could be used on low volume serum samples, and 3) total sample volume available. Some we already know to be associated with renal disfunction. We undertook a literature review specifically focusing on studies published between 2008-2012 reviewing available papers where the abstract indicated association with prevalent or incident nephropathy. We then undertook a round of shortlisting to identify the markers of greatest interest, aiming to include markers across a range of potential mechanisms (eg inflammatory markers, markers related to the glycocalyx). A final selection of 30 biomarkers was made on the basis of this feedback and the practicalities of the ideal combination of markers that could be measured within the limited volume of serum available.

Biomarkers were measured using three differing methods: levels of five biomarkers were measured using single ELISAs and commercially available kits, an extensive panel of biomarkers including 144 identified entities was measured using LS/MS to measure. 58 biomarkers were measured using multiplexed, microsphere-based assays performed using the xMAP multiplexing assaying technology, available from Luminex Corporation (Austin, Tex., UK) (xMAP is a trade mark of Luminex Corporation).

Accordingly, a large set of biomarkers including low and high level metabolites was measured. Multiple reaction monitoring (MRM) mode tryptic digest proteomics was used to give a semi-quantitative measure of a series of targeted peptides. The LS/MS procedure identified around further 137 biomarkers which are as yet not formally characterized. The full list of the 207 biomarkers measured and the lab methods used are set out in the following table. We included serum creatinine in our panel as the eGFRs used to define cases and controls were calculated based on serum creatinine measured clinically and by re-measuring creatinine at baseline we now have a uniform measure of creatinine in all samples.

TABLE 5
Biomarkers analysed, including assay details and QC measures

| | | | | | Intra-assay | Inter-assay |
|---|---|---|---|---|---|---|
| | | | Lower Limit | Upper Limit | Coefficient | Coefficient |
| | | | of | of | of | of |
| Full Name | Assay | Units | Quantitation | Quantitation | Variation | Variation |
| Growth Derived | Elecsys GDF15 | pg/ml | 0 | 20000 | 2.33% | 1.30%-1.54% |
| Factor 15 | (Roche Diagnostics) | | | | | |
| High Sensitivity | Elecsys troponin T | pg/ml | 3 | 10000 | 2.31% | 2.27%-6.60% |
| Troponin | high sensitive | | | | | |
| | (Roche Diagnostics) | | | | | |
| Hyaluronic acid | Hyaluronic Acid | ng/ml | 12.5 | 3200 | 5.90% | 12.90%-14.40% |
| | Sandwich ELISA | | | | | |
| | (Tebubio) | | | | | |
| Soluble RAGE | Human RAGE | ng/ml | 0 | 5000 | 9.10% | 10.10%-13.10% |
| | ELISA (R&D systems) | | | | | |
| Transforming | Human | pg/ml | 15.6 | 1000 | 40.00% | 32.00% |
| Growth Factor | Transforming | | | | | |
| b3 | Growth factor β3, | | | | | |
| | TGF-β3 ELISA Kit | | | | | |
| | (Holzel) | | | | | |
| Adenosine | ESI-MSMS | nM/l | 5 | 10000 | 2.7-3.8% | 80.9-154% |
| Alanine | ESI-MSMS | uM/l | 1 | 2500 | 3.6-4.0% | 4.9-6.9% |
| Albumin T31* | ESI-MSMS | N/A | 1 | — | — | — |
| Albumin T34* | ESI-MSMS | N/A | 1 | — | — | — |
| Albumin T6* | ESI-MSMS | N/A | 1 | — | — | — |
| Albumin T70* | ESI-MSMS | N/A | 1 | — | — | — |
| Alpha-1 | ESI-MSMS | N/A | 1 | — | — | — |
| Antitrypsin (1)* | | | | | | |
| Alpha-1 | ESI-MSMS | N/A | 1 | — | — | — |
| Antitrypsin (2)* | | | | | | |
| Alpha-1 | ESI-MSMS | N/A | 1 | — | — | — |
| Antitrypsin (3)* | | | | | | |
| Alpha-1-acid | ESI-MSMS | N/A | 1 | — | — | — |
| glycoprotein 1 | | | | | | |
| Peak 1* | | | | | | |
| Alpha-1-acid | ESI-MSMS | N/A | 1 | — | — | — |
| glycoprotein 1 | | | | | | |
| Peak 2* | | | | | | |
| Alpha-1-acid | ESI-MSMS | N/A | 1 | — | — | — |
| glycoprotein 2* | | | | | | |
| Alpha-1- | ESI-MSMS | N/A | 1 | — | — | — |
| antichymotrypsin* | | | | | | |
| Alpha-1-B | ESI-MSMS | N/A | 1 | — | — | — |
| glycoprotein (1)* | | | | | | |
| Alpha-2- | ESI-MSMS | N/A | 1 | — | — | — |
| Macroglobulin* | | | | | | |
| Alpha- | ESI-MSMS | N/A | 1 | — | — | — |
| fetoprotein (2)* | | | | | | |
| Alpha- | ESI-MSMS | N/A | 1 | — | — | — |
| fetoprotein (3)* | | | | | | |
| Anthranilic acid | ESI-MSMS | nM/l | | | | |
| Apolipoprotein | ESI-MSMS | N/A | 1 | — | — | — |
| A1* | | | | | | |
| Apolipoprotein | ESI-MSMS | N/A | 1 | — | — | — |
| A-II pre* | | | | | | |
| Apolipoprotein | ESI-MSMS | N/A | 1 | — | — | — |
| A-Q1* | | | | | | |
| Apolipoprotein | ESI-MSMS | N/A | 1 | — | — | — |
| B100*A46 | | | | | | |
| Apolipoprotein | ESI-MSMS | N/A | 1 | — | — | — |
| B-Q1* | | | | | | |
| Apolipoprotein | ESI-MSMS | N/A | 1 | — | — | — |
| C1* | | | | | | |
| Apolipoprotein | ESI-MSMS | N/A | 1 | — | — | — |
| C2* | | | | | | |
| Apolipoprotein | ESI-MSMS | N/A | 1 | — | — | — |
| C3* | | | | | | |
| Apolipoprotein | ESI-MSMS | N/A | 1 | — | — | — |
| E-Q* | | | | | | |
| Arginine | ESI-MSMS | uM/l | 1 | 500 | 3.7-5.4 | 3.9-6.7 |
| Arginine | ESI-MSMS | N/A | 1 | — | — | — |
| diphosphate* | | | | | | |
| Arginine | ESI-MSMS | nM/l | | | | |
| triphosphate | | | | | | |
| Arigininosuccinic | ESI-MSMS | nM/l | | | | |
| acid | | | | | | |
| Arigininosuccinic | ESI-MSMS | nM/l | | | | |
| anhyrdride | | | | | | |
| Aspartic acid | ESI-MSMS | um/l | 5 | 2500 | 12-56.8 | 7.4-57.4 |
| Asymmetric | ESI-MSMS | nM/l | 20 | 10000 | | |
| Dimethylarginine | | | | | | |
| Beta-2- | ESI-MSMS | N/A | 1 | — | — | — |
| glycoprotein-1* | | | | | | |
| C10: 1- | ESI-MSMS | nM/l | 50 | 30000 | 13.9-18.9% | 6-10.7% |
| acylcarnitine | | | | | | |
| C10- | ESI-MSMS | nM/l | 50 | 30000 | 10.9-17.2% | 5.7-6.3% |
| acylcarnitine | | | | | | |
| C12- | ESI-MSMS | nM/l | 10 | 30000 | 13.4-18.4% | 7.8-13.4% |
| acylcarnitine | | | | | | |
| C14: 1- | ESI-MSMS | nM/l | 80 | 200000 | 6.9-10% | 18.7-22.3% |
| acylcarnitine | | | | | | |
| C14- | ESI-MSMS | nM/l | 30 | 200000 | 11.9-15.6% | 13.1-13.4% |
| acylcarnitine | | | | | | |
| C16: 1-OH- | ESI-MSMS | nM/l | 100 | 40000 | | |
| acylcarnitine | | | | | | |
| C16- | ESI-MSMS | nM/l | 80 | 40000 | 11.3-11.8% | 15.8-17.3% |
| acylcarnitine | | | | | | |
| C16—OH- | ESI-MSMS | nM/l | 100 | 40000 | | |
| acylcarnitine | | | | | | |
| C18- | ESI-MSMS | nM/l | 120 | 40000 | 14.5-17.7% | 32-36.4% |
| acylcarnitine | | | | | | |
| C18—OH- | ESI-MSMS | nM/l | 100 | 40000 | | |
| acylcarnitine | | | | | | |
| C2-acylcarnitine | ESI-MSMS | nM/l | 1 | 80 | 2.3-2.5% | 2.5-3.5% |
| C3-acylcarnitine | ESI-MSMS | nM/l | 1 | 80000 | 5.1-8.9% | 5.6-6.1% |
| C3—DC- | ESI-MSMS | nM/l | 20 | 80000 | | 56.4-67.5% |
| acylcarnitine | | | | | | |
| C4-acylcarnitine | ESI-MSMS | nM/l | 1 | 80000 | 6.4-11.0% | 6.8-7.9% |
| C4—DC- | ESI-MSMS | nM/l | 50 | 80000 | 25.5-29.6% | 15.6-19.4% |
| acylcarnitine | | | | | | |
| C5-acylcarnitine | ESI-MSMS | nM/l | 40 | 80000 | 9.8-10.4% | 5.1-11.3% |
| C5—DC- | ESI-MSMS | nM/l | 50 | 100000 | 16.1-19.2% | 12.5-14.6% |
| acylcarnitine | | | | | | |
| C6-acylcarnitine | ESI-MSMS | nM/l | 10 | 40000 | 20.1-29.1% | 13.7-14.5% |
| C6—DC- | ESI-MSMS | nM/l | 50 | 40000 | | 10.4-44.4% |
| acylcarnitine | | | | | | |
| C8-acylcarnitine | ESI-MSMS | nM/l | 25 | 40000 | 18.2-23.9% | 2.2-9.0% |
| Carnosine | ESI-MSMS | nM/l | | | | |
| Ceruloplasmin (1)* | ESI-MSMS | N/A | 1 | — | — | — |
| Ceruloplasmin (2)* | ESI-MSMS | N/A | 1 | — | — | — |
| Ceruloplasmin (3)* | ESI-MSMS | N/A | 1 | — | — | — |
| Citrulline | ESI-MSMS | uM/l | 1 | 500 | 5.20% | 3.8-4.4% |
| Coagulation | ESI-MSMS | N/A | 1 | — | — | — |
| factor IX pre* | | | | | | |
| Coagulation | ESI-MSMS | N/A | 1 | — | — | — |
| factor V (1)* | | | | | | |
| Coagulation | ESI-MSMS | N/A | 1 | — | — | — |
| factor V (2)* | | | | | | |
| Coagulation | ESI-MSMS | N/A | 1 | — | — | — |
| factor XII heavy | | | | | | |
| chain* | | | | | | |
| Complement C2* | ESI-MSMS | N/A | 1 | — | — | — |
| Complement C3 | ESI-MSMS | N/A | 1 | — | — | — |
| (1)* | | | | | | |
| Complement C3 | ESI-MSMS | N/A | 1 | — | — | — |
| (2)* | | | | | | |
| Complement C4 | ESI-MSMS | N/A | 1 | — | — | — |
| beta* | | | | | | |
| Complement C4 | ESI-MSMS | N/A | 1 | — | — | — |
| gamma* | | | | | | |
| Creatine | ESI-MSMS | uM/l | 1 | 250 | 7.1-10.1% | 5.6-7.7% |
| Creatinine | ESI-MSMS | mmol/l | 1 | 1000 | 6.2-8.6% | 4.9-9.1% |
| Dihydrothymin e | ESI-MSMS | uM/l | | | | |
| Dihydrouracik | ESI-MSMS | uM/l | | | | |
| E-selectin (1)* | ESI-MSMS | N/A | 1 | — | — | — |
| Ferritin heavy | ESI-MSMS | N/A | 1 | — | — | — |
| chain* | | | | | | |
| Free carnitine | ESI-MSMS | uM/l | 1 | 200 | 2.4-7.0% | 2.8-3.1% |
| Glutamic acid | ESI-MSMS | uM/l | 1 | 2000 | 9.7-10.8% | 21.7-47.8% |
| Glutamine | ESI-MSMS | um/l | 1->5 | 1000 | 8.6-8.9% | 19.8-88.9% |
| Glycine | ESI-MSMS | uM/l | 15 | 10000 | 8.2-9.6% | 7.7-9.0% |
| Guanidinoacetate | ESI-MSMS | uM/l | | | | |
| Haptoglobin | ESI-MSMS | N/A | 1 | — | — | — |
| beta-chain* | | | | | | |
| Hemopexin (1)* | ESI-MSMS | N/A | 1 | — | — | — |
| Hemopexin (2)* | ESI-MSMS | N/A | 1 | — | — | — |
| Homovanillic | ESI-MSMS | nM/l | | | | |
| acid | | | | | | |
| Hydroxykynurenine | ESI-MSMS | nM/l | | | | |
| Hydroxyproline | ESI-MSMS | uM/l | 1 | 2500 | 21.9-33.0% | 11.5-14.7% |
| Hydroxytryptophan | ESI-MSMS | nM/l | | | | |
| Hypoxanthine | ESI-MSMS | uM/l | | | | |
| Inosine | ESI-MSMS | nM/l | 20 | 10000 | 9.1-10.5% | 48.9-61.4% |
| Isoleucine | ESI-MSMS | uM/l | 5 | 10000 | 17.5-23.8% | 4.5-7.8% |
| Kynurenic acid | ESI-MSMS | nM/l | | | | |
| Kynurenine | ESI-MSMS | uM/l | | | | |
| L-3-O-Methyl | ESI-MSMS | nM/l | | | | |
| Dopa | | | | | | |
| Leucine | ESI-MSMS | uM/l | 1 | 2500 | 9.3-11.1% | 5.1-5.5% |
| Leucine-rich | ESI-MSMS | N/A | 1 | — | — | — |
| alpha-2- | | | | | | |
| glycoprotein* | | | | | | |
| Lysine | ESI-MSMS | uM/l | 1 | 1000 | 7.9-9.1% | 5.1-6.5% |
| Methionine | ESI-MSMS | uM/l | 1 | 600 | 8.2-11.0% | 2.8-3.3% |
| Methylmalonic | ESI-MSMS | nM/l | | | | |
| acid | | | | | | |
| N- | ESI-MSMS | nM/l | | | | |
| acetylaspartate | | | | | | |
| N-Acetyl-BetaD- | ESI-MSMS | uM/l/min | 0.1->2 | 150 | 2.8-5.0% | 5.30% |
| Glucosaminidase | | | | | | |
| Neopterin | ESI-MSMS | nM/l | | | | |
| Nitrotyrosine | ESI-MSMS | nM/l | | | | |
| Ornithine | ESI-MSMS | uM/l | 1 | 500 | 8.4-9.9% | 4.7-7.6% |
| Orotic acid | ESI-MSMS | nM/l | 90 | 10000 | 6.6-6.9% | 35.4-37.7% |
| OXO-2- | ESI-MSMS | nM/l | | | | |
| Deoxyguanosine | | | | | | |
| Phenylalanine | ESI-MSMS | uM/l | 1 | 1000 | 4.6-10.5% | 2.3-3.8% |
| Plasminogen | ESI-MSMS | N/A | 1 | — | — | — |
| Peak 1 (1)* | | | | | | |
| Plasminogen | ESI-MSMS | N/A | 1 | — | — | — |
| Peak 1 (2)* | | | | | | |
| Plasminogen | ESI-MSMS | N/A | 1 | — | — | — |
| Peak 2 (1)* | | | | | | |
| Plasminogen | ESI-MSMS | N/A | 1 | — | — | — |
| Peak 2 (2)* | | | | | | |
| Plasminogen* | ESI-MSMS | N/A | 1 | — | — | — |
| Proline | ESI-MSMS | uM/l | 1 | 650 | 4.2-4.5% | 3.1-3.3% |
| Pyridoxal-5- | ESI-MSMS | nM/l | | | | |
| phosphate | | | | | | |
| Pyroglutamic | ESI-MSMS | nM/l | | | | |
| acid | | | | | | |
| Retinol binding | ESI-MSMS | N/A | 1 | — | — | — |
| protein (1)* | | | | | | |
| Sarcosine | ESI-MSMS | uM/l | | | | |
| Serine | ESI-MSMS | nM/l | 10 | 5000 | 10.6-19.2% | 10.8-12.1% |
| Serotransferrin (1)* | ESI-MSMS | N/A | 1 | — | — | — |
| Serotransferrin (2)* | ESI-MSMS | N/A | 1 | — | — | — |
| Sialic acid | ESI-MSMS | um/l | 0.01 | 25 | 3.0-4.5% | 3.4-5.2% |
| Symmetric | ESI-MSMS | nM/l | 20 | 10000 | 3.7-4.6% | 3.9-11.9% |
| Dimethylarginine | | | | | | |
| Symmetric | ESI-MSMS | — | — | — | — | — |
| Dimethylarginine: | | | | | | |
| Asymmetric | | | | | | |
| Dimethylarginine | | | | | | |
| Taurine | ESI-MSMS | uM/l | 15 | 10000 | 14.2-21.6% | 7.2-8.1% |
| Threonine | ESI-MSMS | uM/l | 1 | 2000 | 5.0-7.5% | 3.5-3.7% |
| Thymine | ESI-MSMS | nM/l | | | | |
| Transferrin (1)* | ESI-MSMS | N/A | 1 | — | — | — |
| Transferrin | ESI-MSMS | N/A | 1 | — | — | — |
| (2)*A82 | | | | | | |
| Transferrin (3)* | ESI-MSMS | N/A | 1 | — | — | — |
| Transthyretin (1)* | ESI-MSMS | N/A | 1 | — | — | — |
| Transthyretin (2)* | ESI-MSMS | N/A | 1 | — | — | — |
| Tryptophan | ESI-MSMS | uM/l | 1 | 500 | | |
| Tyrosine | ESI-MSMS | uM/l | 1 | 600 | | |
| Uracil | ESI-MSMS | nM/l | | | | |
| Ureidobutyrate | ESI-MSMS | nM/l | | | | |
| Ureidopropionate | ESI-MSMS | nM/l | | | | |
| Uric acid | ESI-MSMS | um/l | 10 | 1000 | 2.0-3.4% | 2.6-3.2% |
| Valine | ESI-MSMS | uM/l | 1 | 600 | 6.0-7.6% | 2.6-3.8% |
| Vitamin Dbinding | ESI-MSMS | N/A | 1 | — | — | — |
| (2)* | | | | | | |
| Vitamin Dbinding | ESI-MSMS | N/A | 1 | — | — | — |
| (3)* | | | | | | |
| Vitronectin* | ESI-MSMS | N/A | 1 | — | — | — |
| Von Willebrand | ESI-MSMS | N/A | 1 | — | — | — |
| factor (1)* | | | | | | |
| Von Willebrand | ESI-MSMS | N/A | 1 | — | — | — |
| factor (2)* | | | | | | |
| Xanthine | ESI-MSMS | um/l | 0.01 | 50 | 3.1-5.7% | 16.3-23.9% |
| Xanthurenic acid | ESI-MSMS | um/l | | | | |
| Zinc alpha 2- | ESI-MSMS | N/A | 1 | — | — | — |
| glycoprotein (1)* | | | | | | |
| Adiponectin | Microsphere assay | ug/ml | 0.06 | | 0-14% | 3-8% |
| Adrenomedulli n | Microsphere assay | ng/ml | 0.22 | | 0-15% | 6-8% |
| Alpha-1- | Microsphere assay | ug/ml | 0.12 | | 1-17% | 5-13% |
| Microglobulin | | | | | | |
| Alpha-2- | Microsphere assay | mg/ml | 0.017 | | 0-6% | 6-12% |
| Macroglobulin | | | | | | |
| Apolipoprotein | Microsphere assay | ug/ml | 74 | | 2-20% | 14-37% |
| D | | | | | | |
| Apolipoprotein | Microsphere assay | ug/ml | 4.9 | | 1-12% | 10-20% |
| E | | | | | | |
| Apolipoprotein(a) | Microsphere assay | ug/ml | 4.5 | | 0-9% | 12-16% |
| Beta Amyloid 1-40 | Microsphere assay | ng/ml | 0.0045 | | 0-7% | 8-14% |
| Beta Amyloid 1-42 | Microsphere assay | ng/ml | 0.17 | | 0-13% | 5-16% |
| Beta-2- | Microsphere assay | ug/ml | 0.01 | | 0-14% | 11-13% |
| Microglobulin | | | | | | |
| Calbindin | Microsphere assay | ng/ml | 4.5 | | 2-19% | 8-12% |
| Clusterin | Microsphere assay | ug/ml | 4.8 | | 1-10% | 8-21% |
| C-Reactive | Microsphere assay | ug/ml | 0.039 | | 0-8% | 9-16% |
| Protein | | | | | | |
| Cystatin-C | Microsphere assay | ng/ml | 37 | | 0-12% | 7-16% |
| EN-RAGE | Microsphere assay | ng/ml | 0.22 | | 0-12% | 3-10% |
| Eotaxin-2 | Microsphere assay | pg/ml | 49 | | 0-12% | 5-16% |
| E-Selectin | Microsphere assay | ng/ml | 0.21 | | 0-5% | 3-6% |
| Fatty Acid- | Microsphere assay | ng/ml | 15 | | 0-10% | 5-14% |
| Binding Protein | | | | | | |
| heart | | | | | | |
| Ferritin | Microsphere assay | ng/ml | 4.3 | | 0-15% | 6-15% |
| Fibroblast | Microsphere assay | ng/ml | 0.017 | | 0-11% | 9-14% |
| Growth Factor 21 | | | | | | |
| Fibroblast | Microsphere assay | ng/ml | 0.048 | | 1-9% | 7-11% |
| growth factor 23 | | | | | | |
| Glutathione S- | Microsphere assay | ng/ml | 2.9 | | 0-22% | 10-18% |
| Transferase | | | | | | |
| alpha | | | | | | |
| Heat-Shock | Microsphere assay | ng/ml | 1.5 | | 0-7% | 8-11% |
| protein 70 | | | | | | |
| Interferon | Microsphere assay | pg/ml | 127 | | 0-8% | 8% |
| gamma Induced | | | | | | |
| Protein 10 | | | | | | |
| Interleukin-2 | Microsphere assay | pg/ml | 420 | | 0-8% | 2-11% |
| receptor alpha | | | | | | |
| Interleukin-6 | Microsphere assay | ng/ml | 0.027 | | 0-9% | 5-12% |
| receptor | | | | | | |
| Kidney Injury | Microsphere assay | ng/ml | 0.015 | | 0-14% | 8-15% |
| Molecule-1 | | | | | | |
| Latency- | Microsphere assay | ng/ml | 0.13 | | 0-10% | 6-8% |
| Associated | | | | | | |
| Peptide of | | | | | | |
| Transforming | | | | | | |
| Growth Factor | | | | | | |
| beta 1 | | | | | | |
| Lectin-Like | Microsphere assay | ng/ml | 0.9 | | 0-21% | 4-11% |
| Oxidized LDL | | | | | | |
| Receptor 1 | | | | | | |
| Macrophage | Microsphere assay | pg/ml | 30 | | 0-8% | 4-11% |
| Inflammatory | | | | | | |
| Protein-3 alpha | | | | | | |
| Malondialdehyde- | Microsphere assay | ng/ml | 245 | | 0-14% | 15-18% |
| Modified | | | | | | |
| Low-Density | | | | | | |
| Lipoprotein | | | | | | |
| Methylglyoxal | Microsphere assay | ng/ml | 178 | | 1-20% | 8-18% |
| Methylmalonic | ESI-MSMS | nM/L | — | — | — | — |
| acid | | | | | | |
| MHC class I | Microsphere assay | pg/ml | 73 | | 1-13% | 5-13% |
| chain-related | | | | | | |
| protein A | | | | | | |
| Monocyte | Microsphere assay | pg/ml | 6.7 | | 0-7% | 6-11% |
| Chemotactic | | | | | | |
| Protein 2 | | | | | | |
| Monocyte | Microsphere assay | pg/ml | 445 | | 1-18% | 7-12% |
| Chemotactic | | | | | | |
| Protein 4 | | | | | | |
| Monokine | Microsphere assay | pg/ml | 110 | | 0-11% | 10-20% |
| Induced by | | | | | | |
| Gamma | | | | | | |
| Interferon | | | | | | |
| Myeloid | Microsphere assay | ng/ml | 0.14 | | 0-21% | 4-15% |
| Progenitor | | | | | | |
| Inhibitory Factor 1 | | | | | | |
| Myeloperoxidase | Microsphere assay | ng/ml | 128 | | 1-13% | 5-16% |
| Myoglobin | Microsphere assay | ng/ml | 8.1 | | 1-11% | 5-9% |
| Neutrophil | Microsphere assay | ng/ml | 2.9 | | 0-9% | 2-12% |
| Gelatinase- | | | | | | |
| Associated | | | | | | |
| Lipocalin | | | | | | |
| N-terminal | Microsphere assay | pg/ml | 76 | | 0-5% | 5-7% |
| prohormone of | | | | | | |
| brain natriuretic | | | | | | |
| peptide | | | | | | |
| Osteopontin | Microsphere assay | ng/ml | 3.1 | | 0-17% | 9-20% |
| Osteoprotegeri n | Microsphere assay | pM/l | 0.45 | | 0-16% | 7-15% |
| Plasminogen | Microsphere assay | ng/ml | 3.1 | | 0-12% | 5-9% |
| Activator | | | | | | |
| Inhibitor 1 | | | | | | |
| Secreted | Microsphere assay | ng/ml | 0.47 | | 2-17% | 8-17% |
| frizzled-related | | | | | | |
| protein 4 | | | | | | |
| Serum Amyloid | Microsphere assay | ug/ml | 0.072 | | 0-8% | 4-10% |
| P-Component | | | | | | |
| Sex Hormone- | Microsphere assay | nmol/l | 5.1 | | 1-11% | 7-14% |
| Binding Globulin | | | | | | |
| Tamm-Horsfall | Microsphere assay | ug/ml | 0.00057 | | 0-13% | 14-20% |
| Urinary | | | | | | |
| Glycoprotein | | | | | | |
| T-Cell-Specific | Microsphere assay | ng/ml | 0.21 | | 0-9% | 10-16% |
| Protein RANTES | | | | | | |
| Thrombomodulin | Microsphere assay | ng/ml | 0.24 | | 1-11% | 6-12% |
| Thrombospondin-1 | Microsphere assay | ng/ml | 92 | | 0-10% | 14% |
| Thyroxine- | Microsphere assay | ug/ml | 0.18 | | 0-13% | 5-15% |
| binding globulin | | | | | | |
| Tissue Inhibitor of | Microsphere assay | ng/ml | 3.2 | | 0-11% | 4-8% |
| Metalloproteinases 1 | | | | | | |
| Trefoil Factor 3 | Microsphere assay | ug/ml | 0.0052 | | 3-18% | 7-16% |
| Tumor necrosis | Microsphere assay | ng/ml | 1.1 | | 0-10% | 5-9% |
| factor receptor 2 | | | | | | |
| Tumor Necrosis | Microsphere assay | pg/ml | 36 | | 0-8% | 5-8% |
| Factor Receptor I | | | | | | |
| Vascular Cell | Microsphere assay | ng/ml | 11 | | 1-10% | 3-8% |
| Adhesion | | | | | | |
| Molecule-1 | | | | | | |
| von Willebrand | Microsphere assay | ug/ml | 17 | | 0-18% | 10-17% |
| Factor | | | | | | |

Table 5 also includes a further limited number of biomarkers (Growth Derived Factor 15, Troponin T, Hyaluronic acid, Soluble receptor for Advanced Glycation Endproducts and transforming growth factor beta 3) measured by ELISA.

Two complementary approaches were applied to biomarker selection: forward selection using logistic regression, and sparse logistic regression with the L1 (LASSO) regularization penalty (Tibshirani R. Regression shrinkage and selection via the Lasso. Journal of the Royal Statistical Society Series B-Methodological. 1996; 58(1): 267-88). For the baseline model we used the clinical covariates likely to be available at the randomisation stage of a clinical trial: age, sex, albuminuria, HbA1c, eGFR, ACE Inhibitor use and ARB use. The area under the Receiver Operating Characteristic (AUROC) curve was used as performance metric. All models were developed using nested cross-validation with final models cross-validated.

Details of LC-MSMS Procedure:

Two protocols were used to yield quantitative and semi-quantitative information on 144 metabolites and peptides and proteins with LC-MSMS. The method has been validated against known standards for the entities reported as being quantified (other signals are reported but for as yet unidentified and un-validated entities some of which show clear associations with eGFR). For high sensitivity serum metabolite estimation 30 μL of aqueous standards, controls and samples were pipetted into 1.8 mL polypropylene snap-top Eppendorf tubes. To each tube, 75 μL of methanol/water stable isotope mixture 1 followed by 75 μL of pure methanol was added, the tubes capped, vortex mixed for 2-5 seconds, and centrifuged at 21,000 g at 4° C. for 6 min. Supernatants, 120 μL were transferred to a 96 deep well (2 mL) polypropylene sample block, sealed, and placed in the autosampler at 8° C. ready for analysis by LC electrospray MSMS on an API5500 under Analyst 1.5.2 control (Analyst Software is available from Ab Sciex, Framingham, USA. Analyst is a trade mark of Sciex), Sample supernatants (3 μL) were injected automatically and chromatography was performed on an Astec Chirobiotic T HPLC column 25 cm×2.1 mm, 5 μm (available from Sigma-Aldrich, Missouri, USA) with a 2 cm×4.0 mm, 5 μm guard column with an isocratic running solvent (acetonitrile:water, 1:1, with 0.025% formic acid) at a flow rate of 250 μl/min (Chirobiotic is a trade mark of Sigma-Aldrich). Data was acquired in positive ion MRM (multiple reaction monitoring) mode for 15 min and this was followed by re-injection and data acquisition in negative ion MRM mode for 9 min. For Serum tryptic peptide targeted proteomic analysis, 10 μL of plasma controls and samples were pipetted as above. To each tube, 40 μl of water, 50 μL of diluted stable isotope labelled albumin T6 aqueous internal standard, 10 μL of acetonitile and 10 μL of 1% formic acid were added and mixed on an orbital shaker at RT for 5 min. Then 6 μL of 1M NH₄CO₃ was added to each tube and vortex mixed for 5 seconds before addition of 25 μL of trypsin, vortex mixing, and incubation at 37° C. for 1 h. After incubation, 200 μL of running buffer (acetonitrile:water, 1:1, with 0.025% formic acid) was added to each tube, vortex mixed for 2-5 seconds and centrifuged at 21,000 g at 4° C. for 5 min. The supernatants, 200 μL, were transferred to a 96 deep well (2 mL) polypropylene sample block as above. Sample supernatants (5 μL) were injected automatically and chromatography performed on two, in series, Astec Chirobiotic T HPLC Guard columns 2 cm×4.0 mm, 5 μm with an isocratic running solvent (acetonitrile:water, 1:1, with 0.025% formic acid) at a flow rate of 320 μl/min. Data was acquired in positive ion MRM mode for 10 min. Data was analysed in Analyst version 1.5.2 and MultiQuant version 2.1 (Multi-Quant is available from and is a trade mark of Ab Sciex, Framingham, USA).

Details of the Myriad RBM (MRBM) Platform:

Multiplexed, microsphere-based assays based on the xMAP multiplexing assaying technology (Luminx Corporation, Austin, Tex.) and combining assays for sets of biomarkers on a single chip (Myriad RBM, Austin, Tex.), were performed in a single reaction vessel by combining optical classification schemes, biochemical assays, flow cytometry and digital signal processing hardware and software. (Multiplexing is accomplished by assigning each analyte-specific assay a microsphere set labelled with a unique fluorescence signature. To attain distinct microsphere signatures, two fluorescent dyes, red and far red, are mixed in various combinations using various intensity levels of each dye. Each batch or set of microspheres is encoded with a fluorescent signature by impregnating the microspheres with one of these dye combinations. After the encoding process, an assay-specific capture reagent (i.e., antigens, antibodies, receptors, peptides, enzyme substrates, etc.) is conjugated covalently to each unique set of microspheres. Covalent attachment of the capture reagent to the microspheres is achieved with standard carbodiimide chemistry. After optimizing the parameters of each assay separately, Multi-Analyte Profiles are performed by mixing different sets of the microspheres in a single well of a 96- or 384-format microtiter plate. A small sample volume was added to the well and allowed to react with the microspheres. The assay-specific capture reagent on each individual microsphere binds the analyte of interest. A cocktail of assay-specific, biotinylated detecting reagents (e.g., antigens, antibodies, ligands, etc.), was reacted with the microsphere mixture, followed by a streptavidin labelled fluorescent “reporter” molecule (typically phycoerythrin). Because the microspheres are in suspension, the assay kinetics are near solution-phase. Finally, the multiplex was washed to remove unbound detecting reagents. After washing, the mixture of microspheres was analyzed using the Luminex 100/200™ instrument. Similar to a flow cytometer, the instrument uses hydrodynamic focusing to pass the microspheres in single file through two laser beams. As each individual microsphere passes through the excitation beams, it is analyzed for size, encoded fluorescence signature and the amount of fluorescence generated in proportion to the analyte. The resulting data stream is interpreted using proprietary data analysis software developed at Myriad RBM (Austin, Tex., USA). Assays are run in high density multiplexed panels and the Least Detectable Dose (LDD) was determined as the mean+3 standard deviations of 20 blank readings. The LLOQ was determined by the concentration of an analyte where the measurement of analyte demonstrates a coefficient of variation (CV) of 30%. It represents the lowest concentration of analyte that can be measured with a precision better than or equal to 30%. Appropriate dilutions were made to ensure a quantitative measurement within the limits of the assay. An eight (n=8) point standard curve (S1-S8) was used to obtain quantitative measurements for each sample. Quality Controls (QC's) are run in duplicate along different points of the curve to ensure both accuracy and precision for each analyte.

Clinical Covariates

HbA1c and serum creatinine were measured on the day of sampling as part of routine clinical testing by standard clinical laboratory methods. Albuminuria status was based on highest measures of urinary microalbumin recorded in the clinical record in the 5 years prior to sampling. The standard cut-points of <20 mg/ml for normoalbuminuria, 20-200 mg/ml for microalbuminuria and >200 mg/ml for macroalbuminuria were used. Where no microalbumin was measured the status was set as normoalbuminuric. The use of ACE Inhibitors or Angiotensin Receptor Blockers (ARBs) was determined using primary care prescription data.

Statistical Methods

Data Cleaning and Imputation

The data from the biomarker assays was cleaned and imputed before analysis. Twenty eight biomarkers had either >80% of their measures below the detection threshold or had >80% overall missingness and these biomarkers were therefore dropped from all analysis. Adenosine had <100 samples with measures above the level of detection of the assay so is presented as an ordered categorical variable consisting of the levels—below detection limit, above detection limit but below median and above median. A novel and previously unreported sparse iterative regression model was used for imputation of biomarker measurements missing either because they were below the detection thresholds or because they were missing at random. Each missing measurement was repeatedly predicted from the observed and inputted values using L1-regularized linear and generalized linear models and a correction for the measurement's prior distribution. The flat prior was used for imputing the values missing at random and bounded Pareto priors for imputing continuous censored values (which helped to avoid extraneous spikes at the detection limits. The set of clinical covariates was imputed first (including in sample creatinine and Cystatin C). The remaining biomarkers were imputed using all available data, including imputed clinical covariates, in the reconstruction of the missing values, as well as information concerning the missingness type and the lower and upper detection limits provided by the biomarker assays. This approach allow for the simultaneous imputations of biomarker measurements which are censored because of hardware and/or missing at random and it is a necessary prerequisite for identification of panels of biomarkers using feature selection methods.

Data Analysis

Two complementary approaches were used for biomarker selection: forward selection using logistic regression, and sparse logistic regression with the L1 (LASSO) regularization penalty (Tibshirani R. Regression shrinkage and selection via the Lasso. Journal of the Royal Statistical Society Series B-Methodological. 1996; 58(1): 267-88). Both approaches used adjustments for clinical covariates (age, sex, HbA1C, eGFR calculated using the Modification of Diet in Renal Disease 4-variable (MDRD4) equation, albuminuria and use of ACE inhibitors or ARBs).

Prior to selection models we included two filter steps. In step one all biomarkers that had a correlation of >0.9 were identified and for each pair a single biomarker was retained. The second step used the training set data and identified biomarkers with univariate association with the outcome and selected the 50 biomarkers with the strongest associations. Prediction in models was assessed where we included or omitted this second filtration step and found that the best performance was seen with the filtered models.

The forward selection method started with an initial panel of predictors including the clinical covariates listed above, and kept adding biomarkers, one at a time, to the panel by ranking them according to a measure of relative improvement in the predictive performance. The selection criterion was based on the significance of the increase in conditional likelihood of logistic regression on validation datasets, where 30-inner fold cross-validation was used at each iteration. Significance of the improvement due to a biomarker was assessed by paired one-sided t-tests, where the paired samples were validation likelihoods of the models with and without the biomarker for the fixed validation folds. The selection process terminated when there was no further increment in validation log likelihood greater than or equal to 0.5.

The other approach which was used was based on sparse logistic regression that uses all covariates simultaneously. This alternative approach identified a subset of markers by imposing sparsity-inducing priors on parameters of classification/regression models. The L1 (LASSO) regularization was used, which is one of the best explored approaches in high-dimensional statistics and machine learning (Tibshirani R. Regression shrinkage and selection via the Lasso. Journal of the Royal Statistical Society Series B-Methodological. 1996; 58(1): 267-88). The sparsity hyper-parameter was learned by 30-fold cross-validation. To identify a subset of non-redundant protein markers that are predictive of rapid progression of renal decline independently of the clinical factors, it was assumed that the clinical factors were not penalized, i.e. they were always retained in the resulting sets of covariates. This also facilitates comparisons with biomarker panels obtained by forward selection.

Forward selection and sparse logistic regression were applied to Gaussianized data. Gaussianization transforms continuous random variables to have normally distributed marginal distributions, which leads to better density estimates than common models in many datasets (Chen S S B, Gopinath R A. Gaussianization. Advances in Neural Information Processing Systems 13. 2001; 13: 423-9) and may enable common parametric models to be more robust to errors caused by extreme values.

To assess performance of the biomarker panels produced by forward selection and sparse logistic regression, nested cross-validation was used with 50 outer folds. Test data from the outer folds was used purely for assessing predictive quality of resulting biomarker panels and had not been touched either for learning model parameters/hyper-parameters, or for identifying the panels. The area under the ROC curve (AUROC) was used on test data as the performance criterion. The highest-scoring method was re-applied to select the final biomarker panel using the complete dataset, and summary statistics of the resulting biomarkers were reported.

All data preparation and analyses were performed using R version 2.15.2 (R Development Core Team. R: A language and environment for statistical computing. Vienna, Austria: R Foundation for Statistical Computing; 2013).

Methodology for Addressing Bottlenecks in Biomarker Identification

The most useful methods for high-dimensional predictions utilize the idea of sparsity, where candidate predictors are automatically discarded unless the data confirms that they improve predictions. However, applications of these methods for biomarker identification and risk assessment are limited, because: by learning to discard irrelevant covariates, (a) the methods tend to underestimate (“over-shrink”) the effects of the useful biomarkers, which reduces the quality of predictions; (b) they are not applicable when biomarker measurements may be missing due to a number of random and systematic causes—which is nearly always the case in high-dimensional biomarker predictions; (c) they are mostly applicable for predictions of simple endpoints (i.e. in situations when the cases and controls are well-separated by generalized linear models).

We have overcome all three practical bottlenecks of sparse predictive methods for the prediction of complex endpoints. We hypothesize that the over-shrinking problem (a) is caused by trying to discard irrelevant markers from the panel, and trying to combine the retained markers at the same time. We have solved it by focusing the selection method on a reduced set of candidate markers selected by researchers skillful in the art and combined, when necessary, with pre-filtering methods. This two-layer feature selection procedure reduces the hypothesis space and decouples the selection of individually predictive biomarkers from identifying a non-redundant biomarker combination. Because the procedure uses the prior knowledge, it can be applied even when the sample size is small compared with the size of the hypothesis space, as is the case in the proposed study. We have demonstrated that this procedure leads to a substantial reduction of redundancy and/or improvement in the quality of predictions.

We have solved problem (b) by proposing a novel approach for imputing biomarker measurements missing because of a mixture of disparate causes (e.g. missing at random or missing because the concentrations cannot be detected by the measurement hardware). This approach avoids extraneous modes at the detection thresholds (produced, for example, by some of the commercially available packages) that are the most likely causes of errors, and helps to overcome the problem of the limited sample size of the complete case analysis.

We have addressed problem (c) by identifying a clinically relevant endpoint that was likely to be more amenable to predictions using the existing datasets. Specifically, focusing the analysis on the prediction of rapid progression of diabetic nephropathy rather than the case/control status has resulted in the approximately linear separation of the extreme cases. Hence our approach addresses the practical bottlenecks of the common methods for biomarker identification.

Results and Conclusions

Thirty six of the 207 biomarkers measured were significantly associated in univariate cross-validated logistic regression models with progression of renal dysfunction. Using nested 50 fold cross-validation the predictive performance of a series of biomarker panels identified by forward selection and top down approaches was assessed. It was found that the best “sparse” biomarker panel (with <15 biomarkers) for improving prediction was the panel identified by forward selection, while prediction was maximised by use of a more extensive panel of 34 biomarkers identified using the top down approach.

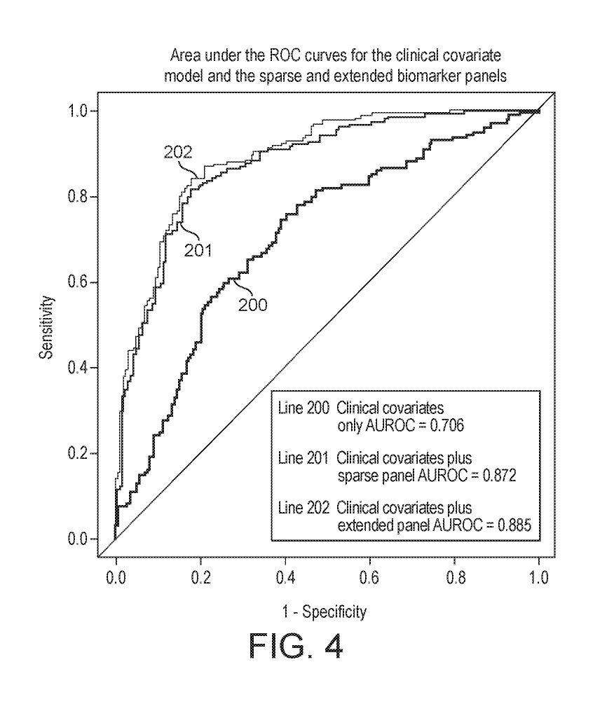

The improvement in prediction for the two panels in comparison to the clinical covariate model alone is shown in FIG. 4. While the clinical covariates (200) alone have an AUROC of 0.706, addition of the sparse panel (201) increased this to 0.872 and addition of the extended panel (202) further increased the AUROC to 0.885. This demonstrates that the biomarker panels can substantially improve the prediction of risk of rapid renal failure in comparison with the clinical covariates alone.

The biomarkers included in the two panels are shown in Tables 1 and 3 above. The 14 biomarkers selected by forward selection and their association with rapid progression adjusted for each other and clinical covariates in a logistic regression model are shown in Tables 1 and 2. While not all biomarkers in the panel have a significant odds ratio (OR) in the logistic regression models the presence of each biomarker increases the predictive power of the overall model.

These biomarkers are for the most part a subset of the maximally predictive 35 biomarker panel selected by top down regression, and shown in Tables 3 and 4, with the exception that cystatin-C was selected with top down, whereas beta-2-microglobulin was selected instead in forward selection. The very high correlation between these biomarkers means that they are quite inter-changeable. Also adrenomedullin showed only weak association with progression when adjusted for the other biomarkers and was not selected in the top down selection.

The 7 biomarkers selected in the forward selection panel on top of the extended clinical covariates are a subset of the 14 biomarker panel—KIM-1, SDMA:ADMA ratio, beta 2-microglobulin, alpha-1 antitrypsin (2), C-16 acylcarnitine, fibroblast growth factor-21 (FGF-21) and uracil. Thus, in the presence of more extensive clinical covariate data these biomarkers may be sufficient to improve prediction.

Accordingly, two novel panels of non-redundant biomarkers have been produced whose performance has been characterized and which significantly improve the prediction of rapid progression of decline in renal function over and above clinical covariates. This is of value as allowing better discrimination of people at high risk of rapid renal decline will allow the enrichment of clinical trials with such people and thus potential savings in the efficiency and cost of these trials.

The novel combination of biomarkers improves the prediction of rapid progression in renal decline among individuals with diabetes. This is of interest as identifying individuals at greatest risk for rapid decline is of benefit not only in a clinical setting but also may greatly improve the power of clinical trials allowing reduction in both the number of people required to be recruited but also the length of follow-up required. At present trials recruit based on known risk factors such as existing renal function and albuminuria but if the trials could be enriched for individuals who will progress over the course of a few years the number of individuals needed to take part in the study would be reduced with potential major reductions in the cost of running the trial.

Importantly, the use of the biomarkers improves the prediction of rapid progression of renal function over models including only baseline clinical covariates including eGFR. This shows the potential utility for some or all of these markers in identifying individuals at greatest risk for rapid decline in renal function.

It is notable that serum creatinine was included in the expanded panel despite inclusion of eGFR (typically based upon measurements of serum creatinine) as a covariate indicating the benefit of a uniformly assayed measure of creatinine over previous clinical measures especially those based on older assays. The majority of our baseline samples were collected prior to the adoption of IDMS-referenced creatinine assays. However, for some the switch in assay will have occurred during the course of follow-up. As the IDMS-referenced creatinine values tend to be lower than those using the older assays 10 this is, if anything, going to have meant we have missed cases rather than artificially resulting in someone meeting the criteria for caseness.

The biomarkers included in the two panels include some that are already well established as associated with renal function such as Cystatin C but also includes more novel associations such as C-16 acylcarnitine and Fibroblast Growth Factor 21.

Additional Information

Measures of 179 biomarkers in 307 individuals were included (153 Cases, 154 Controls) along with the ratio of two biomarkers (Symmetric Dimethylarginine (SDMA): Assymetric Dimethyarginine (ADMA)). For cases the median change in eGFR was −45.2% (IQR −50.5, −42.0) over a median follow up period of 1.9 years (IQR 1.2, 2.6). For controls the median change in eGFR at the end of follow-up was +16.5% (IQR +4.9, +32.4%) over a median follow-up of 5.8 years (IQR 5.5, 6.2). Despite cases and controls all having CKD3 a baseline (i.e. an eGFR between 30-60 ml/min/1.73 m2) the cases had a lower median eGFR at baseline (48.21 ml/min/1.73 m2 vs. 51.34 ml/min/1.73 m2). In addition the cases were on average older, with longer duration of diabetes, and more likely to be male, ex-smokers, with a history of any retinopathy and with an episode of micro or macroalbuminuria in the past five year than the controls (Table 7).

The new measure of serum creatinine was strongly correlated with the recorded creatinine measured at the time of sampling (r=0.89). However, it remained significantly associated with caseness even after adjustment for baseline eGFR. This is not surprising since eGFR measurements were calculated using the serum creatinine measured on the day of sampling by standard clinical assays of the time (primarily alkaline picrate based methods at the time of the study recruitment which are known to over-estimate the true creatinine value (PART 5. EVALUATION OF LABORATORY MEASUREMENTS FOR CLINICAL ASSESSMENT OF KIDNEY DISEASE. KDOQI Clinical Practice Guidelines for Chronic Kidney Disease: Evaluation, Classification, and Stratification 2002, available from: http://www.kidney.org/professionals/kdoqi/guidelinesckd/p5labg4.htm).

There is a high level of correlation between many of the markers included (data not shown). Where two markers were highly correlated (>0.9) we selected a single biomarker from the pair to include in models (data not shown). The median concentrations of measured biomarkers are shown in Table 5 above for both cases and controls and the association of each marker singly was assessed in a series of logistic regression models adjusted for all clinical covariates (Table 5). Many biomarkers showed evidence for association with rapid progression of renal dysfunction with 46 having a p-value<0.01.

Biomarker Selection Panels

A filtering step was used to reduce the number of biomarkers to analyse. This was done in a filtering step where the top 50 biomarkers associated with risk of rapid progression were selected. This was done in the training folds prior to the fitting of the forward selection or top down models. The following Table 6 shows the 50 biomarkers retained after the filtering step and their association with risk of rapid progression. The table is ordered by median of the difference in the test log likelihood for each marker across all test folds. Again there is a high number of markers showing univariate association with progression of renal dysfunction with 46 having a median difference in test log likelihood of >2 which is equivalent to a p-value of <0.05.

TABLE 6

| | Median | | | | | |
|---|---|---|---|---|---|---|
| | difference in | Inter Quartile Range | | | Inter Quartile | |
| | test log | of difference in test | | Median | Range of | |
| Fullname | likelihood | log likelihood | | p-value | p-value | |
| Beta-2-Microglobulin | 13.52 | −0.03, | 16.23 | 0.079 | 0.034, | 0.508 |
| Cystatin-C | 9.48 | 4.92, | 16.90 | 0.054 | 0.015, | 0.289 |
| Trefoil Factor 3 | 9.32 | 5.78, | 12.08 | 0.05 | 0.040, | 0.111 |
| Apolipoprotein D | 9.17 | −0.08, | 17.95 | 0.077 | 0.013, | 0.519 |
| Alpha-1-Microglobulin | 8.53 | 0.43, | 15.57 | 0.071 | 0.019, | 0.408 |
| Adrenomedullin | 8.48 | 6.53, | 9.33 | 0.063 | 0.044, | 0.157 |
| C16-acylcarnitine | 8.37 | 7.77, | 8.95 | 0.053 | 0.045, | 0.096 |
| Growth Derived Factor 15 | 8.25 | 7.21, | 9.25 | 0.088 | 0.074, | 0.124 |
| High Sensitivity Troponin | 7.85 | −0.13, | 9.56 | 0.067 | 0.042, | 0.528 |
| Tissue Inhibitor of | 7.46 | 2.38, | 10.05 | 0.092 | 0.051, | 0.265 |
| Metalloproteinases 1 | | | | | | |
| Kidney Injury Molecule-1 | 6.79 | 2.15, | 8.86 | 0.099 | 0.048, | 0.269 |
| Alpha-1 Antitrypsin (2) | 6.6 | 4.06, | 8.90 | 0.125 | 0.071, | 0.197 |
| Tumor Necrosis Factor | 6.6 | 5.70, | 10.76 | 0.065 | 0.046, | 0.099 |
| Receptor I | | | | | | |
| Interleukin-2 receptor alpha | 6.11 | 3.71, | 7.88 | 0.135 | 0.081, | 0.224 |
| Vascular Cell Adhesion | 6.03 | 4.33, | 7.68 | 0.111 | 0.055, | 0.178 |
| Molecule-1 | | | | | | |
| C2-acylcarnitine | 5.62 | 2.10, | 6.65 | 0.148 | 0.080, | 0.340 |
| Fatty Acid-Binding Protein | 5.62 | −0.06, | 9.58 | 0.198 | 0.074, | 0.517 |
| heart | | | | | | |
| Tumor necrosis factor | 5.52 | 4.45, | 7.58 | 0.12 | 0.069, | 0.181 |
| receptor 2 | | | | | | |
| Methylmalonic acid | 5.25 | 3.25, | 6.37 | 0.153 | 0.085, | 0.217 |
| Tamm-Horsfall Urinary | 4.84 | 3.00, | 7.81 | 0.154 | 0.081, | 0.237 |
| Glycoprotein | | | | | | |
| Lysine | 4.37 | 1.43, | 6.50 | 0.16 | 0.092, | 0.311 |
| Osteoprotegerin | 4.32 | 2.51, | 5.73 | 0.174 | 0.115, | 0.251 |
| N-acetylaspartate | 3.96 | 2.95, | 5.35 | 0.203 | 0.151, | 0.240 |
| Thrombomodulin | 3.89 | 2.01, | 8.13 | 0.167 | 0.061, | 0.280 |
| Fibroblast Growth Factor 21 | 3.87 | −0.07, | 7.57 | 0.242 | 0.061, | 0.517 |
| Osteopontin | 3.85 | 2.76, | 5.71 | 0.192 | 0.128, | 0.250 |
| N-terminal prohormone of | 3.7 | 1.66, | 4.68 | 0.217 | 0.162, | 0.289 |
| brain natriuretic peptide | | | | | | |
| Leucine-rich alpha-2- | 3.5 | 2.61, | 7.28 | 0.22 | 0.124, | 0.321 |
| glycoprotein | | | | | | |
| Von Willebrand Factor | 3.44 | 1.18, | 5.85 | 0.192 | 0.108, | 0.342 |
| Symmetric | 3.43 | 0.54, | 5.86 | 0.214 | 0.090, | 0.396 |
| Dimethylarginine: Asymmetric | | | | | | |
| Dimethylarginine | | | | | | |
| Uracil | 2.76 | 0.14, | 3.87 | 0.262 | 0.184, | 0.477 |
| Thymine | 2.57 | 1.62, | 4.59 | 0.241 | 0.138, | 0.304 |
| C3-DC-acylcarnitine | 2.56 | 0.73, | 4.22 | 0.266 | 0.173, | 0.415 |
| Symmetric | 2.53 | 1.46, | 3.66 | 0.26 | 0.194, | 0.312 |
| Dimethylarginine | | | | | | |
| Sialic acid | 2.48 | 1.07, | 3.95 | 0.298 | 0.171, | 0.370 |
| Tryptophan | 2.44 | 1.14, | 4.43 | 0.219 | 0.156, | 0.388 |
| Latency-Associated Peptide of | 1.77 | −1.13, | 5.85 | 0.257 | 0.117, | 0.856 |
| Transforming Growth Factor | | | | | | |
| beta 1 | | | | | | |
| Monokine Induced by Gamma | 1.74 | 0.34, | 4.82 | 0.267 | 0.168, | 0.523 |
| Interferon | | | | | | |
| C14: 1-acylcarnitine | 1.58 | 0.42, | 2.38 | 0.321 | 0.201, | 0.416 |
| C5-DC-acylcarnitine | 1.4 | 0.53, | 2.84 | 0.32 | 0.260, | 0.412 |
| Creatine | 1.3 | −1.16, | 2.74 | 0.334 | 0.220, | 0.778 |
| Haptoglobin beta-chain | 1.18 | 0.29, | 2.45 | 0.357 | 0.233, | 0.453 |
| Hydroxyproline | 0.63 | −0.92, | 3.78 | 0.428 | 0.220, | 0.978 |
| Ceruloplasmin (2) | 0.58 | −0.40, | 1.87 | 0.402 | 0.292, | 0.583 |
| Hemopexin (2) | 0.51 | −0.77, | 3.14 | 0.421 | 0.231, | 0.759 |
| Fibroblast growth factor 23 | 0.44 | −0.52, | 1.66 | 0.435 | 0.284, | 0.606 |
| C6-DC-acylcarnitine | 0.35 | −0.54, | 2.52 | 0.444 | 0.182, | 0.572 |
| Lectin-Like Oxidized LDL | 0.3 | −0.78, | 1.40 | 0.454 | 0.328, | 0.644 |
| Receptor 1 | | | | | | |
| Transforming Growth Factor | 0.1 | −0.63, | 1.39 | 0.48 | 0.341, | 0.627 |
| b3 | | | | | | |
| Macrophage Inflammatory | −0.89 | −1.66, | 5.10 | 0.803 | 0.144, | 0.983 |
| Protein-3 alpha | | | | | | |

Selected Biomarkers:

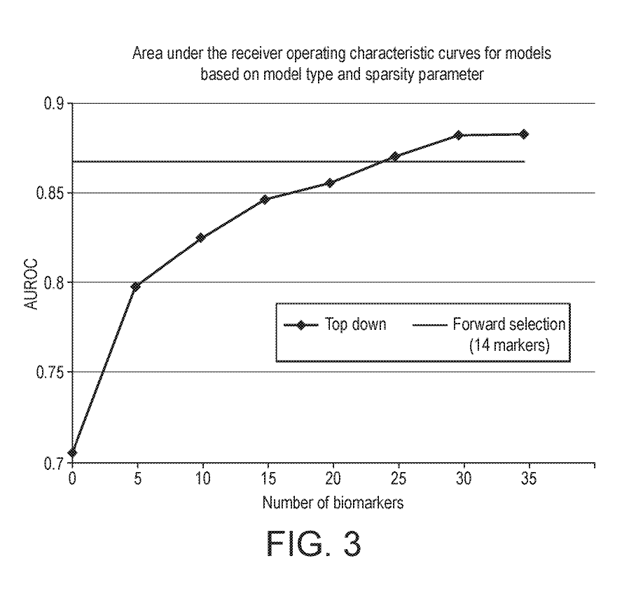

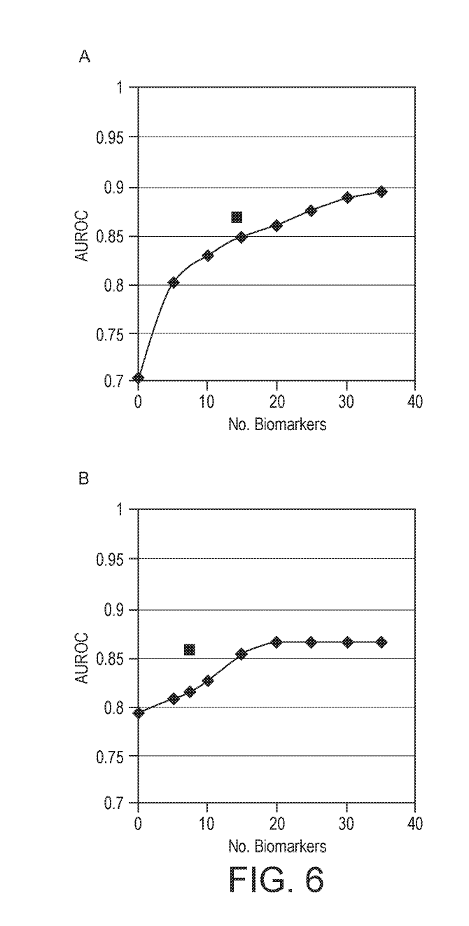

The performance of each model was assessed by calculating AUROCs using nested cross-validated models. The cross-validation allows for more robust estimates with the AUROC more likely to reflect results seen in a new dataset. FIG. 3 shows the differences in AUROC by model type (forward selection or top down), and for the top down models according to the number of biomarkers allowed into each model. Overall the model with the greatest AUROC was the top down restricted clinical covariate model limited to a maximum of 35 biomarkers (AUROC=0.885). The best AUROC for a ‘sparse’ biomarker panel (<15 markers) is achieved by the forward selection model which includes 14 biomarkers. Whilst the best model overall is the top down model restricted to a maximum of 35 biomarkers (AUROC=0.885). FIG. 6 shows the AUROC curves for the sparse and extended biomarker sets in prediction over the baseline covariates models.

Table 2 shows the odds ratios for the best sparse panel of biomarkers and Table 4 shows the odds ratios for the expanded panel. All but two of the biomarkers in the sparse panel (adrenomedullin and beta-2 microglobulin) are featured in the expanded panel. It is important to note that for both panels individual biomarkers are not always statistically significantly associated with progression using the p-value as a criterion but are still contributing to the prediction of the panel. Also for the expanded panel the high degree of correlation and co-linearity between markers makes estimation of a precise odds ratio difficult.

Further Results

Overall 12.5% of the Go-DARTS population with CKD3 at baseline lost >40% of their baseline eGFR within 3.5 years and were therefore defined as cases. Baseline demographics for the study population are shown in the following Table 7. Cases had longer diabetes duration, greater prevalence of albuminuria and retinopathy and a slightly lower median eGFR at baseline than the controls who had stable eGFR during follow up.

TABLE 7
Baseline characteristics for cases and controls

| | Control (n = 154) | | Case (n = 153) | |
|---|---|---|---|---|
| | Median (Inter | | Median (Inter | |
| | Quartile Range)/ | | Quartile Range)/ | |
| | Frequency (%) | | Frequency (%) | |
| Age (years) | 72 | (66, 76) | 74 | (69, 80) |
| Male sex | 55 | (35.7%) | 65 | (42.5%) |
| Diabetes duration (years) | 7.2 | (3.5, 11.0) | 9.1 | (5.1, 15.4) |
| Body Mass Index (kg/m2) | 29.5 | (26.1, 34.4) | 30.6 | (27.1, 34.8) |
| HbA1c (%) | 7.1 | (6.4, 8.2) | 7.3 | (6.5, 8.5) |
| Baseline Estimated Glomerular Filtration | 51.3 | (44.9, 54.6) | 48.2 | (40.5, 54.8) |
| Rate (ml/min) | | | | |
| Weighted Estimated Glomerular Filtration | 57.8 | (52.6, 63.5) | 50.7 | (44.8, 56.7) |
| Rate (ml/min) | | | | |
| Systolic Blood Pressure (mmHg) | 144.3 | (130.0, 153.4) | 144.0 | (131.0, 158.0) |
| Diastolic Blood Pressure (mmHg) | 73.3 | (66.5, 79.4) | 71.0 | (63.5, 78.0) |
| In receipt of Blood Pressure Lowering Drugs | 147 | (95.5%) | 147 | (96.1%) |
| In receipt of ACE inhibitors | 83 | (53.9%) | 100 | (65.4%) |
| In receipt of Angiotensin Receptor Blockers | 30 | (19.5%) | 23 | (15.0%) |
| In receipt of Diuretics | 93 | (60.4%) | 106 | (69.3%) |
| In receipt of Calcium Channel Blocker | 66 | (42.9%) | 56 | (36.6%) |
| In receipt of Beta Blockers | 75 | (48.7%) | 67 | (43.8%) |
| In receipt of Alpha Blockers | 17 | (11.0%) | 21 | (13.7%) |
| On Insulin | 39 | (25.3%) | 47 | (30.7%) |
| Smoking Status | | | | |
| Current smoker | 18 | (11.7%) | 17 | (11.1%) |
| Ex-smoker | 62 | (40.3%) | 89 | (58.2%) |
| Never smoker | 74 | (48.1%) | 47 | (30.7%) |
| Macro or Microalbuminuria | 29 | (18.8%) | 69 | (45.1%) |
| Prior CVD | 33 | (21.4%) | 43 | (28.1%) |
| History of any retinopathy | 85 | (55.2%) | 114 | (74.5%) |
| In sample creatinine (μg/l) | 98.2 | (80.5, 112.6) | 113.1 | (91.2, 127.9) |

Data Reduction Steps

All 207 biomarkers measured are listed in Table 5. From this initial panel we removed forty-two biomarkers from further analysis as being uninformative, either because very few patients had detectable levels of the biomarker n=22, or the biomarker was in tight correlation (r>0.9) with another biomarker (n=15) or because of too few results due to inadequate sample volume analysis (n=5). This left 165 biomarkers and in addition we evaluated the ratio of symmetric dimethylarginine (SDMA) to asymmetric dimethylarginine (ADMA) giving 166 measures. A correlation matrix was calculated for these biomarkers and the continuous clinical covariates. Many of the biomarkers measured had very strong correlations with each other. Of note cystatin C and beta 2-microglobulin were tightly correlated (rho=0.86).

Univariate Associations

The volcano plot of FIG. 3 shows the associations with rapid progression for all 166 biomarkers evaluated singly and adjusted for baseline age, sex, eGFR, albuminuria, HbA1c, and ACE Inhibitor and Angiotensin Receptor Blocker use. Cystatin-C and beta 2-microglobulin had the strongest associations with similar effect sizes per standard deviate. We retained 63 biomarkers for further evaluation as having at least suggestive evidence for association in the initial cross validated logistic regression for each biomarker evaluated alone. The following Table 8 lists the biomarkers from this set of 63 that reached Bonferroni adjusted significance level (p<0.0008) on adjustment for clinical covariates, examined singly.

TABLE 8
Thirty Five Biomarkers Significantly Associated with Rapid Progression of
eGFR Examined Singly and Adjusted for Clinical Covariates.

| | Control | | | | | | |
|---|---|---|---|---|---|---|---|
| | Median | Control IQR | Case Median | Case IQR | OR | 95% Cl | p. value |
| Adrenomedullin | 2.2 | 1.7, 2.6 | 2.9 | 2.4, 3.6 | 2.94 | 2.10, 4.22 | <0.00001 |
| (ng/ml) | | | | | | | |
| Alpha-1 | 161.37 | 127.79, 187.81 | 186.38 | 165.60, 214.51 | 1.77 | 1.36, 2.33 | <0.00001 |
| Antitrypsin | | | | | | | |
| (2) | | | | | | | |
| Alpha-1- | 17.0 | 14.0, 20.0 | 21.0 | 19.0, 24.3 | 3.31 | 2.31, 4.88 | <0.00001 |
| Microglobulin | | | | | | | |
| (ug/ml) | | | | | | | |
| Aspartic acid | 32.97 | 27.20, 37.61 | 34.67 | 31.91, 41.03 | 1.63 | 1.25, 2.15 | 0.00038 |
| (uM/l) | | | | | | | |
| Beta-2- | 2.0 | 1.7, 2.4 | 2.7 | 2.4, 3.4 | 6.11 | 3.90, 10.05 | <0.00001 |
| Microglobulin | | | | | | | |
| (ug/ml) | | | | | | | |
| C16-acylcarnitine | 284.14 | 227.66, 355.38 | 305.26 | 280.32, 395.84 | 1.68 | 1.29, 2.21 | 0.00015 |
| (nM/l) | | | | | | | |
| C18-acylcarnitine | 121.89 | 111.48, 158.46 | 130.83 | 119.71, 204.29 | 1.57 | 1.22, 2.06 | 0.00074 |
| (nM/l) | | | | | | | |
| C2-acylcarnitine | 8.48 | 6.78, 10.44 | 9.34 | 8.49, 11.20 | 1.64 | 1.25, 2.18 | 0.00047 |
| (uM/l) | | | | | | | |
| Creatinine (uM/l) | 98.18 | 80.49, 112.60 | 113.1 | 91.16, 127.90 | 3.43 | 1.97, 6.36 | <0.00001 |
| Cystatin-C (ng/ml) | 1340 | 1140, 1510 | 1680 | 1490, 1900 | 6.34 | 3.88, 10.97 | <0.00001 |
| Fibroblast Growth | 0.25 | 0.16, 0.43 | 0.40 | 0.30, 0.65 | 2.06 | 1.56, 2.80 | <0.00001 |
| Factor 21 (ng/ml) | | | | | | | |
| Fibroblast growth | 0.08 | 0.05, 0.13 | 0.12 | 0.08, 0.22 | 1.85 | 1.37, 2.55 | <0.00001 |
| factor | | | | | | | |
| 23 (ng/ml) | | | | | | | |
| Growth Derived | 2328 | 1761, 3355 | 3785 | 2681, 5555 | 2.30 | 1.69, 3.20 | <0.00001 |
| Factor | | | | | | | |
| 15 (pg/ml) | | | | | | | |
| High Sensitivity | 5.29 | 2.89, 12.89 | 16.53 | 9.78, 26.75 | 3.15 | 2.11, 4.85 | <0.00001 |
| Troponin T (pg/ml) | | | | | | | |
| Interleukin-2 | 2493 | 2075, 3152 | 3174 | 2710, 4180 | 2.45 | 1.79, 3.43 | <0.00001 |
| receptor | | | | | | | |
| alpha (pg/ml) | | | | | | | |
| Kidney Injury | 0.05 | 0.04, 0.08 | 0.09 | 0.07, 0.16 | 2.60 | 1.88, 3.68 | <0.00001 |
| Molecule- | | | | | | | |
| 1 (ng/ml) | | | | | | | |
| Leucine-rich | 136.58 | 115.20, 155.79 | 149.71 | 138.26, 189.60 | 1.64 | 1.25, 2.18 | 0.00047 |
| alpha-2- | | | | | | | |
| glycoprotein | | | | | | | |
| Lysine (uM/l) | 217.67 | 190.63, 244.12 | 203.9 | 174.87, 215.00 | 0.55 | 0.42, 0.72 | <0.00001 |
| Methylmalonic | 270 | 220, 350 | 366 | 310, 460 | 2.09 | 1.56, 2.87 | <0.00001 |
| acid (nM/l) | | | | | | | |
| N-acetylaspartate | 296.58 | 239.50, 378.61 | 341.62 | 306.09, 452.19 | 1.76 | 1.33, 2.37 | 0.00013 |
| (nM/l) | | | | | | | |
| N-terminal | 552.5 | 247.00, 1152.50 | 1487.23 | 607.00, 3170.00 | 2.10 | 1.54, 2.94 | <0.00001 |
| prohormone | | | | | | | |
| of brain natriuretic | | | | | | | |
| peptide (pg/ml) | | | | | | | |
| Osteopontin | 15 | 11, 23 | 26 | 18, 33 | 2.58 | 1.82, 3.78 | <0.00001 |
| Sialic acid (uM/l) | 1.09 | 0.93, 1.31 | 1.37 | 1.19, 1.76 | 2.43 | 1.73, 3.52 | <0.00001 |
| Symmetric | 564 | 499.00, 647.50 | 662.91 | 578.00, 786.00 | 2.49 | 1.72, 3.69 | <0.00001 |
| Dimethylarginine | | | | | | | |
| SDMA:ADMA | 1.06 | (0.93, 1.22) | 1.23 | 1.13, 1.49 | 2.63 | 1.86, 3.81 | <0.00001 |
| Tamm-Horsfall | 0.04 | 0.03, 0.05 | 0.03 | 0.02, 0.03 | 0.46 | 0.33, 0.62 | <0.00001 |
| Urinary | | | | | | | |
| Thrombomodulin | 5.39 | 4.60, 6.40 | 6.5 | 5.75, 7.40 | 2.00 | 1.48, 2.73 | <0.00001 |
| (ng/ml) | | | | | | | |
| Tissue Inhibitor of | 170 | 150, 192 | 188 | 172, 218 | 2.02 | 1.52, 2.74 | <0.00001 |
| Metalloproteinases | | | | | | | |
| 1 (ng/ml) | | | | | | | |
| Trefoil Factor 3 | 0.17 | 0.13, 0.22 | 0.27 | 0.21, 0.37 | 4.17 | 2.81, 6.42 | <0.00001 |
| Tryptophan (uM/l) | 57.31 | 50.65, 64.25 | 52.21 | 43.10, 56.76 | 0.54 | 0.41, 0.72 | <0.00001 |
| Tumor Necrosis | 2639 | 1985, 3217 | 3440 | 2852, 4130 | 2.41 | 1.76, 3.37 | <0.00001 |
| Factor Receptor I | | | | | | | |
| (pg/ml) | | | | | | | |
| Tumor necrosis | 9.7 | 8.30, 12.00 | 13 | 10.00, 16.00 | 2.55 | 1.84, 3.63 | <0.00001 |
| factor receptor 2 | | | | | | | |
| (ng/ml) | | | | | | | |
| Uracil (nM/l) | 119.26 | 94.25, 152.80 | 136.54 | 121.23, 172.55 | 1.76 | 1.35, 2.35 | <0.00001 |
| Vascular Cell | 603 | 530, 747 | 724 | 612, 885 | 1.77 | 1.34, 2.36 | <0.00001 |
| Adhesion | | | | | | | |
| von Willebrand | 82 | 64.25, 110.75 | 98 | 83.13, 133.00 | 1.65 | 1.26, 2.19 | 0.00035 |
| Factor (ug/ml) | | | | | | | |

Biomarker Panel Performance

FIG. 6A shows the performance of the biomarker panels chosen by the forward selection process with a fixed termination criterion and those chosen by a top down selection process that was run with varying sparsity constraints i.e. set to terminate at different numbers of retained biomarkers. The AUROC for a model including only clinical covariates was 0.706. The forward selection process selected 14 of the retained 63 biomarkers as contributing to prediction improvement beyond the clinical covariates. This yielded a substantial increment in AUROC to 0.868. As shown, using the top down approach, performance could be improved to varying degrees depending on the number of biomarkers selected reaching a maximum AUROC of 0.892 but requiring a larger set of 35 biomarkers to achieve this.

The GO-DARTS dataset contains retrospective clinical data for several years pre-baseline so we further examined the contribution of biomarkers to prediction beyond that achieved by using an extended set of clinical covariates data including longitudinal eGFR (see methods for full list) pre-baseline. This extended clinical dataset showed a substantially higher prediction than the basic clinical covariate model (AUROC=0.793 vs. 0.706 respectively). As shown in FIG. 6B addition of selected biomarkers improved prediction somewhat further with an increment in AUROC from 0.793 to 0.859 with a panel of 7 biomarkers selected by forward selection, and a maximal AUROC of 0.868 achieved with top down selection retaining 25 biomarkers. Use of at least 8 of the biomarkers gives a significant improvement to the AUROC without the additional clinical covariates, with a particularly significant increase for at least 10.

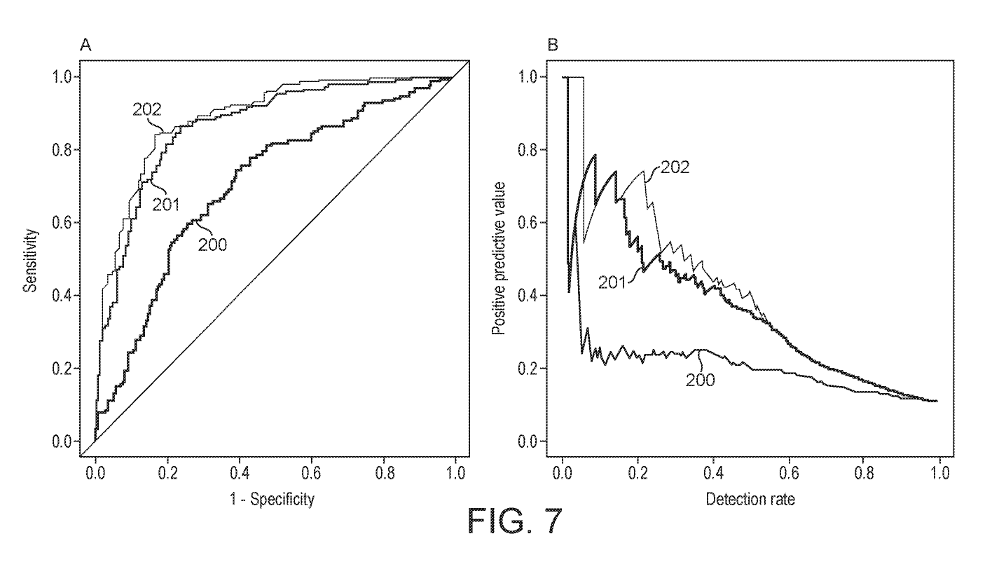

FIG. 7A shows the AUROC curves for the forward selected panel of 14 biomarkers and the top down selection of 35 biomarkers. However, a useful metric that summarises the potential value of biomarkers in selection of patients for a clinical trial is the “predicted event rate enrichment” achieved by using the biomarker panel in a given potential clinical trial population (FIG. 7B). For example In the Go-DARTS cohort just 12.5% of those meeting the eGFR baseline entry criterion of 30-60 ml/min/1.73 m2 progressed to being a case within 3.5 years. Thus, without any selection by risk stratification (y axis of FIG. 7B=1) the expected cumulative incidence of progression is 12.5% (x-axis). The plot illustrates that selecting say the top 20% (y axis=0.2) of patients based on their score from a model combining clinical covariates and selected biomarkers could enrich the cumulative incidence of rapid progression to >60%.

1. A method of determining renal function decline risk in a subject, comprising (a) analysing one or more samples obtained from the subject for the level of each of a plurality of biomarkers, (b) comparing the measured levels of each of the plurality of biomarkers with a respective plurality of control levels, and (c) determining renal function decline risk in the subject from the comparison, wherein the said plurality of biomarkers comprise at least 7 of the group consisting of: (i) adrenomedullin; (ii) alpha-1 antitrypsin; (iii) beta-2-microglobulin or cystatin-C; (iv) c16-acylcarnitine; (v) creatine; (vi) creatinine; (vii) fatty acid-binding protein, heart; (viii) fibroblast growth factor 21; (ix) hydroxyproline; (x) kidney injury molecule-1; (xi) N-terminal prohormone of brain natriuretic peptide; (xii) the ratio of symmetric dimethylarginine to asymmetric dimethylarginine; (xiii) symmetric dimethylarginine; and (xiv) uracil.

2. A method according to claim 1, wherein step (c) also comprises taking into account each of a plurality of clinical covariates concerning the subject, wherein the clinical covariates comprise at least 5 of the group consisting of: (a) the age of the subject; (b) the gender of the subject; (c) an estimate of glomerular filtration rate (eGRF) of the subject; (d) the level of albumin in urine from the patient; (e) the level of HbA1c in one or more samples obtained from the subject; (f) a measure of the use of ACE inhibitors by the subject; (g) a measure of the use of angiotensin receptor blocker use by the subject.

3. A method of selecting a subject as a candidate subject for a study concerning the efficacy of a method of treatment on renal function or the progression of decline in renal function, comprising carrying out the method of claim 1 and determining whether to select the subject as a candidate subject for the study in dependence on the said comparison.

4. A method of selecting a subject for treatment using a therapeutic intervention to maintain, increase, prevent or slow a reduction in renal function, comprising carrying out the method of claim 1 and determining whether to select the subject as a candidate subject for the study in dependence on the said comparison.

5. A method of monitoring the efficacy of a therapeutic intervention on a subject comprising (a) analysing one or more samples obtained from the subject for the level of each of a plurality of biomarkers, (b) comparing the measured levels of each of the plurality of biomarkers with a respective plurality of control levels, (c) determining risk of decline in renal function in the subject from the comparison; (d) administering the therapeutic intervention to the subject, repeating steps (a), (b) and (c), then (e) comparing the determined risk arising from the comparison of the measured levels before the step of administering the therapeutic intervention to the subject with the determined risk arising from the comparison of the measured levels after the step of administering the therapeutic intervention, and (f) assessing the efficacy of the therapeutic intervention in dependence on that said comparison of the determined risks, wherein the said plurality of biomarkers comprise at least 7 of the group consisting of: (i) adrenomedullin; (ii) alpha-1 antitrypsin (iii) beta-2-microglobulin (iv) c16-acylcarnitine; (v) creatine; (vi) creatinine; (XV) cystatin-C; (vii) fatty acid-binding protein, heart; (viii) fibroblast growth factor 21; (ix) hydroxyproline; (x) kidney injury molecule-1; (xi) N-terminal prohormone of brain natriuretic peptide; (xii) the ratio of symmetric dimethylarginine to asymmetric dimethylarginine; (xiii) symmetric dimethylarginine; and (xiv) uracil.

6. A method according to claim 5, wherein step (c) also comprises taking into account each of a plurality of clinical covariates pertaining to the subject, wherein the clinical covariates comprise at least 5 of the group consisting of: (a) the age of the subject; (b) the gender of the subject; (c) an estimate of glomerular filtration rate (eGRF) of the subject; (d) the level of albumin in urine from the patient; (e) the level of HbA1c in one or more samples obtained from the subject; (f) a measure of the use of ACE inhibitors by the subject; (g) a measure of the use of angiotensin receptor blocker use by the subject.

7. A method, carried out by one or more processors of a computer, of processing the levels of a plurality of biomarkers obtained by analysis of one or more samples obtained from the subject, and data concerning the statistical relationship between the levels of said plurality of biomarkers and the risk of renal function decline in the patient, to thereby estimate risk of renal function decline in the subject, wherein the said plurality of biomarkers comprise at least 7 of the group consisting of: (i) adrenomedullin; (ii) alpha-1 antitrypsin; (iii) beta-2-microglobulin; (iv) c16-acylcarnitine; (v) creatine; (vi) creatinine; (vii) fatty acid-binding protein, heart; (viii) fibroblast growth factor 21; (ix) hydroxyproline; (x) kidney injury molecule-1; (xi) N-terminal prohormone of brain natriuretic peptide; (xii) the ratio of symmetric dimethylarginine to asymmetric dimethylarginine; (xiii) symmetric dimethylarginine; and (xiv) uracil.

8. A method according to claim 7, wherein method further comprises processing a plurality of clinical covariates concerning the same subject and the said data concern the statistical relationship between the levels of said plurality of biomarkers and said plurality of clinical covariates, wherein the clinical covariates comprise at least 5 of the group consisting of: (a) the age of the subject; (b) the gender of the subject; (c) an estimate of glomerular filtration rate (eGRF) of the subject; (d) the level of albumin in urine from the patient; (e) the level of HbA1c in one or more samples obtained from the subject; (f) a measure of the use of ACE inhibitors by the subject; (g) a measure of the use of angiotensin receptor blocker use by the subject.

9. A method according to claim 1, wherein the said plurality of biomarkers comprise at least 12 of the group consisting of: (i) adrenomedullin; (ii) alpha-1 antitrypsin; (iii) beta-2-microglobulin; (iv) c16-acylcarnitine; (v) creatine; (vi) creatinine; (vii) fatty acid-binding protein, heart; (viii) fibroblast growth factor 21; (ix) hydroxyproline; (x) kidney injury molecule-1; (xi) N-terminal prohormone of brain natriuretic peptide; (xii) the ratio of symmetric dimethylarginine to asymmetric dimethylarginine; (xiii) symmetric dimethylarginine; and (xiv) uracil.

10. A method according to claim 1, wherein the said plurality of biomarkers comprises or consists of the group consisting of: (i) adrenomedullin; (ii) alpha-1 antitrypsin; (iii) beta-2-microglobulin; (iv) c16-acylcarnitine; (v) creatine; (vi) creatinine; (vii) fatty acid-binding protein, heart; (viii) fibroblast growth factor 21; (ix) hydroxyproline; (x) kidney injury molecule-1; (xi) N-terminal prohormone of brain natriuretic peptide; (xii) the ratio of symmetric dimethylarginine to asymmetric dimethylarginine; (xiii) symmetric dimethylarginine; and (xiv) uracil.

11. A method according to claim 1, wherein said plurality of biomarkers further comprises (xv) cystatin-C.

12. A method according to claim 1, wherein the said plurality of biomarkers includes (vi) creatinine but the estimate of (c) glomerular filtration rate is also based on a measure of creatinine in a sample obtained from the subject.

13. A method according to claim 1, wherein the plurality of biomarkers does not comprise adrenomedullin and/or does not comprise beta-2-microglobulin.

14. A method according to claim 1, wherein the plurality of biomarkers comprises (viii) fibroblast growth factor 21.

15. A method according to claim 1, wherein the plurality of biomarkers comprises (iv) c16-acylcarnitine.

16. A method according to claim 1, wherein the plurality of biomarkers comprises (ix) hydroxyproline.

17. A method according to claim 1, wherein the plurality of biomarkers comprises (vii) fatty acid-binding protein heart.

18. A method according to claim 1, wherein the plurality of biomarkers comprises (v) creatine.

19. A method according to claim 1, wherein the said plurality of biomarkers comprises or consists of the group consisting of (x) kidney injury molecule-1, (xii) the ratio of symmetric dimethylarginine to asymmetric dimethylarginine, (iii) beta 2-microglobulin, (ii) alpha-1 antitrypsin, (iv) C-16 acylcarnitine, (viii) fibroblast growth factor-21 (FGF-21) and (xiv) uracil.

20. A method according to claim 19, wherein step (c) also comprises taking into account each of a plurality of clinical covariates pertaining to the subject, wherein the plurality of clinical covariates comprises at least 8, at least 10, or comprising or consisting of all of the group consisting of: (a) the age of the subject; (b) the gender of the subject; (c) an estimate of glomerular filtration rate (eGFR) of the subject; (d) the level of albumin in urine from the patient; (e) the level of HbA1c in one or more samples obtained from the subject; (f) a measure of the use of ACE inhibitors by the subject; (g) a measure of the use of angiotensin receptor blocker use by the subject, (h) a measured of the subject's body mass index, (i) a measure of the duration of the subject's diabetes condition, (j) a measure of the subject's systolic blood pressure, and (k) a measure of the subject's diastolic blood pressure

21. A method according to claim 1, wherein the said plurality of biomarkers comprises at least 20 of the group consisting of: (ii) alpha-1 antitrypsin; (iv) c16-acylcarnitine; (v) creatine; (vi) creatinine; (vii) fatty acid-binding protein, heart; (viii) fibroblast growth factor 21; (ix) hydroxyproline; (x) kidney injury molecule-1; (xi) N-terminal prohormone of brain natriuretic peptide; (xii) the ratio of symmetric dimethylarginine to asymmetric dimethylarginine; (xiii) symmetric dimethylarginine; (xiv) uracil; (xv) cystatin-C; (xvi) apolipoprotein D or e-selectin; (xvii) fibroblast growth factor 23; (xviii) glutamic acid; (xix) haptoglobin beta-chain; (xx) troponin; (xxi) hypoxanthine; (xxiii) interleukin-2 receptor alpha; (xxiv) latency-associated peptide of transforming growth factor beta 1; (xxv) leucine-rich alpha-2-glycoprotein; (xxvi) lysine; (xxvii) monokine induced by Gamma Inteferon; (xxviii) methylmalonic acid; (xxix) N-acetylaspartate; (xxx) neutrophil gelatinase-associated lipocalin; (xxxi) osteopontin; (xxxii) Tamm-Horsfall urinary glycoprotein; (xxxiii) thymine; (xxxiv) tissue inhibitor of metalloproteinases 1; (xxxvi) tryptophan; (xxxvii) tumour necrosis factor receptor 1; (xxxviii) von Willebrand factor.

22. A method according to claim 21, wherein the said plurality of biomarkers comprises or consists of the said group consisting of: (ii) alpha-1 antitrypsin; (iv) c16-acylcarnitine; (v) creatine; (vi) creatinine; (vii) fatty acid-binding protein, heart; (viii) fibroblast growth factor 21; (ix) hydroxyproline; (x) kidney injury molecule-1; (xi) N-terminal prohormone of brain natriuretic peptide; (xii) the ratio of symmetric dimethylarginine to asymmetric dimethylarginine; (xiii) symmetric dimethylarginine; (xiv) uracil; (xv) cystatin-C; (xvi) apolipoprotein D or e-selectin; (xvii) fibroblast growth factor 23; (xviii) glutamic acid; (xix) haptoglobin beta-chain; (xx) troponin; (xxi) hypoxanthine; (xxiii) interleukin-2 receptor alpha; (xxiv) latency-associated peptide of transforming growth factor beta 1; (xxv) leucine-rich alpha-2-glycoprotein; (xxvi) lysine; (xxvii) monokine induced by Gamma Inteferon; (xxviii) methylmalonic acid or monomethylarsonous acid; (xxix) N-acetylaspartate; (xxx) neutrophil gelatinase-associated lipocalin; (xxxi) osteopontin; (xxxii) Tamm-Horsfall urinary glycoprotein; (xxxiii) thymine; (xxxiv) tissue inhibitor of metalloproteinases 1; (xxxvi) tryptophan; (xxxvii) tumour necrosis factor receptor 1; (xxxviii) von Willebrand factor.

23. A method according to claim 1, wherein the level of the ratio of symmetric dimethylarginine to asymmetric dimethylarginine is expressed as the ratio of measurements of the level of symmetric dimethylarginine and the level of asymmetric dimethylarginine in any suitable units.

24. A method according to claim 23, wherein the plurality of biomarkers comprises both (xiii) symmetric dimethylarginine and (xii) the ratio of symmetric dimethylarginine to assymetric dimethylarginine and an increased level of symmetric dimethylarginine is indicative of a reduced risk of renal function decline and an increased ratio of symmetric dimethylarginine to assymetric dimethylarginine is indicative of an increased risk of renal function decline.

25. A method according to claim 1, wherein the determined risk is a value indicative of a level of risk, or one of a finite group of risk levels, or a binary value.

26. A method according to claim 1, wherein the level of at least one biomarker is measured by measuring a product derived from the biomarker.

27. A method of determining renal function decline risk in a subject, comprising (a) analysing one or more samples obtained from the subject for the level of one or more biomarkers including fibroblast growth factor 21, (b) comparing the measured levels of each of the one or more plurality of biomarkers with a respective one or more control levels, and (c) determining renal function decline risk in the subject from the comparison, wherein an increased level of fibroblast growth factor 21 may be correlated with an increased risk of renal function decline.

28. A method of determining renal function decline risk in a subject, comprising (a) analysing one or more samples obtained from the subject for the level of one or more biomarkers including c16-acylcarnitine, (b) comparing the measured levels of each of the one or more plurality of biomarkers with a respective one or more control levels, and (c) determining renal function decline risk in the subject from the comparison, wherein an increased level of c16-acylcarnitine is correlated with an increased risk of renal function decline.
